(19) United States
(12) Patent Application Publication
(10) Pub. No.: US 2021/0155715 A1
(43) Pub. Date: May 27, 2021

(54) USE OF TRYPTOPHAN DERIVATIVES AND L-METHIONINE FOR PROTEIN FORMULATION

(71) Applicant: Genentech, Inc., South San Francisco, CA (US)

(72) Inventors: Cleo SALISBURY, South San Francisco, CA (US); Vikas SHARMA, South San Francisco, CA (US); Sreedhara ALAVATTAM, South San Francisco, CA (US)

(21) Appl. No.: 17/168,621

(22) Filed: Feb. 5, 2021

Related U.S. Application Data
(63) Continuation of application No. PCT/US2019/045420, filed on Aug. 7, 2019.
(60) Provisional application No. 62/716,239, filed on Aug. 8, 2018.

(51) Int. Cl. C07K 16/42

(52) U.S. Cl. CPC C07K 16/42

(57) ABSTRACT

The present disclosure provides methods and formulations comprising a polypeptide comprising solvent accessible amino acid residues susceptible to oxidation wherein N-acetyl-DL-tryptophan (NAT) and/or L-methionine is used to prevent oxidation of the polypeptide.

### CROSS-REFERENCE TO RELATED APPLICATIONS

This application claims the benefit of U.S. Provisional Application No. 62/716,239, filed Aug. 8, 2018, each of which is hereby incorporated by reference in its entirety.

### FIELD OF THE INVENTION

The present disclosure relates to liquid formulations comprising a polypeptide, N-acetyl-DL-tryptophan, and L-methionine, and methods for their production and use.

### BACKGROUND

The bioactivity of therapeutic proteins, including monoclonal antibodies (mAbs), depends on conformational and biochemical stability. Oxidation is one of many degradation concerns in therapeutic protein development because it may negatively impact pharmacokinetics or biological activity, particularly if oxidation occurs in regions of the protein involved in binding to the physiological target, or in regions critical to effector function. Additionally, oxidation may alter the susceptibility of a therapeutic protein to aggregation with consequent impact to the immunogenicity profile.

A common solution for the management of oxidation risk in bio therapeutics is lyophilization. However, this approach is not always desirable because it may increase the cost of production, and may make the manufacturing and clinical use of the drug more complex. Protein re-engineering via mutation of oxidation-prone amino acid residues is also a possible approach to mitigate oxidation risk. However, targeted mutations are not always a viable solution because, while they may decrease the likelihood of oxidation, they may also decrease the binding affinity of the protein for its target and, consequently, the potency of the protein. Thus, there is need for alternative or complementary strategies for controlling therapeutic protein oxidation during manufacture, storage, and use.

Examples of polypeptide formulations are disclosed in WO 2010/030670, WO 2014/160495, WO 2014/160497 and WO 2017/117304.

All references cited herein, including patent applications, patent publications, non-patent literature, and UniProtKB/Swiss-Prot/GenBank Accession numbers are herein incorporated by reference in their entirety, as if each individual reference were specifically and individually indicated to be incorporated by reference.

### BRIEF SUMMARY

To meet the above and other needs, disclosed herein are liquid formulations comprising a polypeptide (e.g., a therapeutic polypeptide such as an antibody), N-acetyl-DL-tryptophan (NAT), and L-methionine, where the NAT and L-methionine are provided in amounts sufficient to reduce or prevent oxidation of one or more amino acid residues (e.g., tryptophan residues, methionine residues, etc.) in the polypeptide. The present disclosure is based, at least in part, on the finding that, while the addition of NAT was effective at protecting variable region tryptophan residues of two exemplary antibodies during oxidative stress, the inclusion of NAT sensitized Fc methionine residues to oxidation. However, it was found that the addition of L-methionine to formulations comprising NAT effectively protected both tryptophan and methionine residues from oxidation for both of the exemplary antibodies (see Example 1). The present disclosure is also based, at least in part, on the finding that both excipients were well tolerated in vivo (see Example 1), indicating that NAT and L-methionine may be safe and effective as antioxidant excipients in biotherapeutic formulations.

Accordingly, in one aspect, provided herein is a liquid formulation comprising a polypeptide, N-acetyl-DL-tryptophan (NAT), and L-methionine, wherein the NAT is provided in an amount sufficient to prevent oxidation of one or more tryptophan residues in the polypeptide, and wherein the L-methionine is provided in an amount sufficient to prevent oxidation of one or more methionine residues in the polypeptide. In some embodiments, the concentration of NAT in the formulation is about 0.01 to about 25 mM. In some embodiments, the concentration of NAT in the formulation is about 0.05 to about 1.0 mM. In some embodiments, the concentration of NAT in the formulation is about 0.05 to about 0.3 mM. In some embodiments, the concentration of NAT in the formulation is a concentration selected from the group consisting of about 0.05 mM, about 0.1 mM, about 0.3 mM, and about 1.0 mM. In some embodiments, the concentration of L-methionine in the formulation is about 1 to about 125 mM. In some embodiments, the concentration of L-methionine in the formulation is about 5 to about 25 mM. In some embodiments, the concentration of L-methionine in the formulation is about 5 mM. In some embodiments, the concentration of NAT in the formulation is about 0.3 mM and the concentration of L-methionine in the formulation is about 5.0 mM. In some embodiments, the concentration of NAT in the formulation is about 1.0 mM and the concentration of L-methionine in the formulation is about 5.0 mM.

In some embodiments of the invention, the polypeptide is an antibody. In some embodiments, the one or more tryptophan residues of the polypeptide are located within a variable region of the antibody. In some embodiments, the one or more tryptophan residues comprises W103, wherein residue numbering is according to Kabat numbering. In some embodiments, the one or more tryptophan residues are located within an HVR of the antibody. In some embodiments, the one or more tryptophan residues are located within an HVR-H1 and/or an HVR-H3 of the antibody. In some embodiments, the one or more tryptophan residues comprises W33, W36, W52a, W99, W100a, and/or W100b, wherein residue numbering is according to Kabat numbering. In some embodiments, the one or more methionine residues are located within a variable region of the antibody. In some embodiments, the one or more methionine residues comprises M34 and/or M82, wherein residue numbering is according to Kabat numbering. In some embodiments, the one or more methionine residues are located within a constant region of the antibody. In some embodiments, the one or more methionine residues comprises M252 and/or M428, wherein residue numbering is according to EU numbering. In some embodiments, the antibody is an IgG1, IgG2, IgG3, or IgG4 antibody. In some embodiments, the antibody is a polyclonal antibody, a monoclonal antibody, a humanized antibody, a human antibody, a chimeric antibody, a multispecific antibody, or an antibody fragment.

In some embodiments of the invention, the oxidation of the one or more tryptophan residues in the polypeptide is reduced relative to the oxidation of one or more corresponding tryptophan residues in the polypeptide in a liquid formulation lacking NAT. In some embodiments, the oxidation of the one or more methionine residues in the polypeptide is reduced relative to the oxidation of one or more corresponding methionine residues in the polypeptide in a liquid formulation lacking L-methionine. In some embodiments, the oxidation of the one or more tryptophan residues and the one or more methionine residues in the polypeptide is reduced relative to the oxidation of one or more corresponding tryptophan residues and one or more corresponding methionine residues in the polypeptide in a liquid formulation lacking NAT and L-methionine. In some embodiments, the oxidation is reduced by about 40%, about 50%, about 75%, about 80%, about 85%, about 90%, about 95% or about 99%.

In some embodiments of the invention, the polypeptide concentration in the formulation is about 1 mg/mL to about 250 mg/mL. In some embodiments, the formulation has a pH of about 4.5 to about 7.0. In some embodiments, the formulation further comprises one or more excipients. In some embodiments, the one or more excipients are selected from the group consisting of a stabilizer, a buffer, a surfactant, and a tonicity agent.

In some embodiments of the invention, the formulation is a pharmaceutical formulation suitable for administration to a subject. In some embodiments, the pharmaceutical formulation is suitable for subcutaneous, intravenous, or intravitreal administration. In some embodiments, the subject is a human.

In some aspects, the invention provides an article of manufacture or kit comprising the liquid formulation as described herein.

In some aspects, the invention provides a method of reducing oxidation of a polypeptide in an aqueous formulation comprising adding NAT and L-methionine to the formulation, wherein the NAT is provided in an amount sufficient to prevent oxidation of one or more tryptophan residues in the polypeptide, and wherein the L-methionine is provided in an amount sufficient to prevent oxidation of one or more methionine residues in the polypeptide. In some embodiments, the NAT is added to the formulation to a concentration of about 0.01 to about 25 mM. In some embodiments, the NAT is added to the formulation to a concentration of about 0.05 to about 1 mM. In some embodiments, the NAT is added to the formulation to a concentration of about 0.05 to about 0.3 mM. In some embodiments, the NAT is added to the formulation to a concentration selected from the group consisting of about 0.05 mM, about 0.1 mM, about 0.3 mM, and about 1.0 mM. In some embodiments, the L-methionine is added to the formulation to a concentration of about 1 to about 125 mM. In some embodiments, the L-methionine is added to the formulation to a concentration of about 5 to about 25 mM. In some embodiments, the L-methionine is added to the formulation to a concentration of about 5 mM. In some embodiments, the NAT is added to the formulation to a concentration of about 0.3 mM, and wherein the L-methionine is added to the formulation to a concentration of about 5.0 mM. In some embodiments, the NAT is added to the formulation to a concentration of about 1.0 mM, and wherein the L-methionine is added to the formulation to a concentration of about 5.0 mM.

In some embodiments of the invention, the polypeptide is an antibody. In some embodiments, the one or more tryptophan residues of the polypeptide are located within a variable region of the antibody. In some embodiments, the one or more tryptophan residues comprises W103, wherein residue numbering is according to Kabat numbering. In some embodiments, the one or more tryptophan residues are located within an HVR of the antibody. In some embodiments, the one or more tryptophan residues are located within an HVR-H1 and/or an HVR-H3 of the antibody. In some embodiments, the one or more tryptophan residues comprises W33, W36, W52a, W99, W100a, and/or W100b, wherein residue numbering is according to Kabat numbering. In some embodiments, the one or more methionine residues are located within a variable region of the antibody. In some embodiments, the one or more methionine residues comprises M34 and/or M82, wherein residue numbering is according to Kabat numbering. In some embodiments, the one or more methionine residues are located within a constant region of the antibody. In some embodiments, the one or more methionine residues comprises M252 and/or M428, wherein residue numbering is according to EU numbering. In some embodiments, the antibody is an IgG1, IgG2, IgG3, or IgG4 antibody. In some embodiments, the antibody is a polyclonal antibody, a monoclonal antibody, a humanized antibody, a human antibody, a chimeric antibody, a multispecific antibody, or an antibody fragment.

In some embodiments of the invention, the oxidation of the one or more tryptophan residues in the polypeptide is reduced relative to the oxidation of one or more corresponding tryptophan residues in the polypeptide in a liquid formulation lacking NAT. In some embodiments, the oxidation of the one or more methionine residues in the polypeptide is reduced relative to the oxidation of one or more corresponding methionine residues in the polypeptide in a liquid formulation lacking L-methionine. In some embodiments, the oxidation of the one or more tryptophan residues and the one or more methionine residues in the polypeptide is reduced relative to the oxidation of one or more corresponding tryptophan residues and one or more corresponding methionine residues in the polypeptide in a liquid formulation lacking NAT and L-methionine. In some embodiments, the oxidation is reduced by about 40%, about 50%, about 75%, about 80%, about 85%, about 90%, about 95% or about 99%.

In some embodiments of the invention, the polypeptide concentration in the formulation is about 1 mg/mL to about 250 mg/mL. In some embodiments, the formulation has a pH of about 4.5 to about 7.0. In some embodiments, the formulation further comprises one or more excipients. In some embodiments, the one or more excipients are selected from the group consisting of a stabilizer, a buffer, a surfactant, and a tonicity agent. In some embodiments, the formulation is a pharmaceutical formulation suitable for administration to a subject. In some embodiments, the pharmaceutical formulation is suitable for subcutaneous, intravenous, or intravitreal administration. In some embodiments, the subject is a human.

It is to be understood that one, some, or all of the properties of the various embodiments described above and herein may be combined to form other embodiments of the present disclosure. These and other aspects of the present disclosure will become apparent to one of skill in the art. These and other embodiments of the present disclosure are further described by the detailed description that follows.

### BRIEF DESCRIPTION OF THE DRAWINGS

FIGS. 1A-1B show the impact of N-acetyl-DL-tryptophan (NAT) concentration on oxidation levels of two exemplary IgG1 antibodies (mAb1 and mAb2) upon 2,2′-azo-bis(2-amidinopropane) dihydrochloride (AAPH) stress. FIG. 1A shows the impact of NAT concentration on Fv tryptophan oxidation levels. FIG. 1B shows the impact of NAT concentration on Fc methionine oxidation levels.

FIGS. 2A-2B show the oxidation levels after AAPH stress of two exemplary IgG1 antibodies (mAb1 and mAb2) formulated with no methionine or NAT, 5 mM methionine, 0.3 mM NAT, or the combination of 5 mM methionine and 0.3 mM NAT. FIG. 2A shows the oxidation levels of oxidation-sensitive Fv tryptophans. FIG. 2B shows the oxidation levels of Fc methionines.

FIGS. 3A-3B show the impact of NAT concentration on oxidation levels of two exemplary IgG1 antibodies (mAb1 and mAb2) after high-UV light stress. FIG. 3A shows the impact of NAT concentration on HVR tryptophan oxidation levels. FIG. 3B shows the impact of NAT concentration on Fc methionine oxidation levels.

FIGS. 4A-4B show the oxidation levels after high-UV light stress of two exemplary IgG1 antibodies (mAb1 and mAb2) formulated with no methionine or NAT, 5 mM methionine, 0.3 mM NAT, or the combination of 5 mM methionine and 0.3 mM NAT. FIG. 4A shows the oxidation levels of HVR tryptophans. FIG. 4B shows the oxidation levels of Fc methionines.

FIG. 5 shows that anti-oxidants mitigate chemical oxidation risk.

FIG. 6 shows protection from oxidation of W52 with I mM NAT and 5 mM methionine.

### DETAILED DESCRIPTION

### I. Definitions

Before describing the present disclosure in detail, it is to be understood that the present disclosure is not limited to particular compositions or biological systems, which can, of course, vary. It is also to be understood that the terminology used herein is for the purpose of describing particular embodiments only, and is not intended to be limiting.

As used herein, the singular forms “a”, “an” and “the” include plural referents unless the content clearly dictates otherwise. Thus, for example, reference to “a molecule” optionally includes a combination of two or more such molecules, and the like.

The term “about” as used herein refers to the usual error range for the respective value readily known to the skilled person in this technical field. Reference to “about” a value or parameter herein includes (and describes) embodiments that are directed to that value or parameter per se.

It is understood that aspects and embodiments of the present disclosure described herein include “comprising,” “consisting,” and “consisting essentially of” aspects and embodiments.

The term “and/or” as used herein a phrase such as “A and/or B” is intended to include both A and B; A or B; A (alone); and B (alone). Likewise, the term “and/or” as used herein a phrase such as “A, B, and/or C” is intended to encompass each of the following embodiments: A, B, and C; A, B, or C; A or C; A or B; B or C; A and C; A and B; B and C; A (alone); B (alone); and C (alone).

The term “pharmaceutical formulation” refers to a preparation which is in such form as to permit the biological activity of the active ingredient to be effective, and which contains no additional components which are unacceptably toxic to a subject to which the formulation would be administered. Such formulations are sterile.

A “sterile” formulation is aseptic or free or essentially free from all living microorganisms and their spores.

A “stable” formulation is one in which the polypeptide therein essentially retains its physical stability and/or chemical stability and/or biological activity upon storage. Preferably, the formulation essentially retains its physical and chemical stability, as well as its biological activity upon storage. The storage period is generally selected based on the intended shelf-life of the formulation. Various analytical techniques for measuring polypeptide stability are available in the art and are reviewed in Peptide and Protein Drug Delivery, 247-301, Vincent Lee Ed., Marcel Dekker, Inc., New York, N.Y., Pubs. (1991) and Jones, A. Adv. Drug Delivery Rev. 10: 29-90 (1993), for example. Stability can be measured at a selected amount of light exposure and/or temperature for a selected time period. Stability can be evaluated qualitatively and/or quantitatively in a variety of different ways, including evaluation of aggregate formation (for example using size exclusion chromatography, by measuring turbidity, and/or by visual inspection); evaluation of ROS formation (for example by using a light stress assay or a 2,2′-Azobis(2-Amidinopropane) Dihydrochloride (AAPH) stress assay); oxidation of specific amino acid residues of the protein (for example a Trp residue and/or a Met residue of a monoclonal antibody); by assessing charge heterogeneity using cation exchange chromatography, image capillary isoelectric focusing (icIEF) or capillary zone electrophoresis; amino-terminal or carboxy-terminal sequence analysis; mass spectrometric analysis; SDS-PAGE analysis to compare reduced and intact antibody; peptide map (for example tryptic or LYS-C) analysis; evaluating biological activity or target binding function of the protein (e.g., antigen binding function of an antibody); etc. Instability may involve any one or more of: aggregation, deamidation (e.g. Asn deamidation), oxidation (e.g. Met oxidation and/or Trp oxidation), isomerization (e.g. Asp isomeriation), clipping/hydrolysis/fragmentation (e.g. hinge region fragmentation), succinimide formation, unpaired cysteine(s), N-terminal extension, C-terminal processing, glycosylation differences, etc.

A polypeptide “retains its physical stability” in a pharmaceutical formulation if it shows no or very little signs of aggregation, precipitation, fragmentation, and/or denaturation upon visual examination of color and/or clarity, or as measured by, for example, UV light scattering or size exclusion chromatography.

A polypeptide “retains its chemical stability” in a pharmaceutical formulation, if the chemical stability at a given time is such that the polypeptide is considered to still retain its biological activity as defined below. Chemical stability can be assessed by detecting and quantifying chemically altered forms of the polypeptide. Chemical alteration may involve polypeptide oxidation which can be evaluated using, for example, tryptic peptide mapping, reverse-phase high-performance liquid chromatography (HPLC) and liquid chromatography-mass spectrometry (LC/MS). Other types of chemical alteration include charge alteration of the polypeptide which can be evaluated by, for example, ion-exchange chromatography or icIEF.

A polypeptide “retains its biological activity” in a pharmaceutical formulation, if the biological activity of the polypeptide at a given time is within about 20% (such as within about 10%) of the biological activity exhibited at the time the pharmaceutical formulation was prepared (within the errors of the assay), as determined, for example, in an antigen binding assay for a monoclonal antibody.

As used herein, “biological activity” of a polypeptide refers to the ability of the polypeptide to bind its target, for example the ability of a monoclonal antibody to bind to an antigen. It can further include a biological response which can be measured in vitro or in vivo. Such activity may be antagonistic or agonistic.

A polypeptide which is “susceptible to oxidation” is one comprising one or more residue(s) that has been found to be prone to oxidation such as, but not limited to, methionine (Met), cysteine (Cys), histidine (His), tryptophan (Trp), and tyrosine (Tyr). For example, a tryptophan amino acid in the Fab portion of a monoclonal antibody or a methionine amino acid in the Fc portion of a monoclonal antibody may be susceptible to oxidation.

An “oxidation labile” residue of a polypeptide is a residue having greater than 35% oxidation in an oxidation assay (e.g. AAPH-induced or thermal-induced oxidation). The percent oxidation of a residue in a polypeptide can be determined by any method known in the art, such as, for example, tryptic digest followed by LC-MS/MS for site-specific Trp oxidation.

A “solvent-accessible surface area” or “SASA” of a biomolecule in a solvent is the surface area of the biomolecule that is accessible to the solvent. SASA can be expressed in units of measurement (e.g., square Angstroms) or as a percentage of the surface area that is accessible to the solvent. For example, the SASA of an amino acid residue in a polypeptide can be 80 Å², or 30%. SASA can be determined by any method known in the art, including, for example, using the Shrake-Rupley algorithm, the LCPO method, the power diagram method, or molecular dynamics simulations.

The term “isotonic” in reference to a formulation of interest refers to a formulation having essentially the same osmotic pressure as human blood. Isotonic formulations will generally have an osmotic pressure from about 250 to 350 mOsm. Isotonicity can be measured, for example, using a vapor pressure or ice-freezing type osmometer.

As used herein, “buffer” refers to a buffered solution that resists changes in pH by the action of its acid-base conjugate components. For example, a buffer of the present disclosure may have a pH in the range from about 4.5 to about 8.0. Histidine acetate is an example of a buffer that will control the pH in this range.

A “preservative” is a compound which can be optionally included in the formulation to essentially reduce bacterial action therein, thus facilitating the production of a multi-use formulation, for example. Examples of potential preservatives include octadecyldimethylbenzyl ammonium chloride, hexamethonium chloride, benzalkonium chloride (a mixture of alkylbenzyldimethylammonium chlorides in which the alkyl groups are long-chain compounds), and benzethonium chloride. Other types of preservatives include aromatic alcohols such as phenol; butyl and benzyl alcohol, alkyl parabens such as methyl or propyl paraben, catechol, resorcinol, cyclohexanol, 3-pentanol, and m-cresol. In one embodiment, the preservative herein is benzyl alcohol.

As used herein, a “surfactant” refers to a surface-active agent, preferably a nonionic surfactant. Examples of surfactants herein include polysorbate (for example, polysorbate 20 and, polysorbate 80); poloxamer (e.g. poloxamer 188); Triton; sodium dodecyl sulfate (SDS); sodium laurel sulfate; sodium octyl glycoside; lauryl-, myristyl-, linoleyl-, or stearyl-sulfobetaine; lauryl-, myristyl-, linoleyl- or stearyl-sarcosine; linoleyl-, myristyl-, or cetyl-betaine; lauroamidopropyl-, cocamidopropyl-, linoleamidopropyl-, myristamidopropyl-, palmidopropyl-, or isostearamidopropyl-betaine (e.g. lauroamidopropyl); myristamidopropyl-, palmidopropyl-, or isostearamidopropyl-dimethylamine; sodium methyl cocoyl-, or disodium methyl oleyl-taurate; and the MONAQUAT™ series (Mona Industries, Inc., Paterson, N.J.); polyethyl glycol, polypropyl glycol, and copolymers of ethylene and propylene glycol (e.g. Pluronics, PF68 etc); etc. In one embodiment, the surfactant herein is polysorbate 20. In yet another embodiment, the surfactant herein is poloxamer 188.

“Pharmaceutically acceptable” excipients or carriers as used herein include pharmaceutically acceptable carriers, stabilizers, buffers, acids; bases, sugars, preservatives, surfactants, tonicity agents, and the like, which are well known in the art (Remington: The Science and Practice of Pharmacy, 22^{nd} Ed., Pharmaceutical Press, 2012). Examples of pharmaceutically acceptable excipients include buffers such as phosphate, citrate, acetate, and other organic acids; antioxidants including ascorbic acid, L-tryptophan and methionine; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; metal complexes such as Zn-protein complexes; chelating agents such as EDTA; sugar alcohols such as mannitol or sorbitol; salt-forming counterions such as sodium; and/or nonionic surfactants such as polysorbate, poloxamer, polyethylene glycol (PEG), and PLURONICS™. “Pharmaceutically acceptable” excipients or carriers are those which can reasonably be administered to a subject to provide an effective dose of the active ingredient employed and that are nontoxic to the subject being exposed thereto at the dosages and concentrations employed.

The polypeptide which is formulated is preferably essentially pure and desirably essentially homogeneous (e.g., free from contaminating proteins etc.). “Essentially pure” polypeptide means a composition comprising at least about 90% by weight of the polypeptide (e.g. monoclonal antibody), based on total weight of the composition, preferably at least about 95% by weight. “Essentially homogeneous” polypeptide means a composition comprising at least about 99% by weight of the polypeptide (e.g., monoclonal antibody), based on total weight of the composition.

The terms “protein”, “polypeptide”, and “peptide” are used interchangeably herein to refer to polymers of amino acids of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. Examples of polypeptides encompassed within the definition herein include mammalian polypeptides, such as, e.g., renin; a growth hormone, including human growth hormone and bovine growth hormone; growth hormone releasing factor; parathyroid hormone; thyroid stimulating hormone; lipoproteins; alpha-1-antitrypsin; insulin A-chain; insulin B-chain; proinsulin; follicle stimulating hormone; calcitonin; luteinizing hormone; glucagon; leptin; clotting factors such as factor VIIIC, factor IX, tissue factor, and von Willebrands factor; anti-clotting factors such as Protein C; atrial natriuretic factor; lung surfactant; a plasminogen activator, such as urokinase or human urine or tissue-type plasminogen activator (t-PA); bombesin; thrombin; hematopoietic growth factor; tumor necrosis factor-alpha and -beta; a tumor necrosis factor receptor such as death receptor 5 and CD120; TNF-related apoptosis-inducing ligand (TRAIL); B-cell maturation antigen (BCMA); B-lymphocyte stimulator (BLyS); a proliferation-inducing ligand (APRIL); enkephalinase; RANTES (regulated on activation normally T-cell expressed and secreted); human macrophage inflammatory protein (MIP-1-alpha); a serum albumin such as human serum albumin; Muellerian-inhibiting substance; relaxin A-chain; relaxin B-chain; prorelaxin; mouse gonadotropin-associated peptide; a microbial protein, such as beta-lactamase; DNase; IgE; a cytotoxic T-lymphocyte associated antigen (CTLA), such as CTLA-4; inhibin; activin; platelet-derived endothelial cell growth factor (PD-ECGF); a vascular endothelial growth factor family protein (e.g., VEGF-A, VEGF-B, VEGF-C, VEGF-D, and P1GF); a platelet-derived growth factor (PDGF) family protein (e.g., PDGF-A, PDGF-B, PDGF-C, PDGF-D, and dimers thereof); fibroblast growth factor (FGF) family such as aFGF, bFGF, FGF4, and FGF9; epidermal growth factor (EGF); receptors for hormones or growth factors such as a VEGF receptor(s) (e.g., VEGFR1, VEGFR2, and VEGFR3), epidermal growth factor (EGF) receptor(s) (e.g., ErbB1, ErbB2, ErbB3, and ErbB4 receptor), platelet-derived growth factor (PDGF) receptor(s) (e.g., PDGFR-α and PDGFR-β), and fibroblast growth factor receptor(s); TIE ligands (Angiopoietins, ANGPT1, ANGPT2); Angiopoietin receptor such as TIE1 and TIE2; protein A or D; rheumatoid factors; a neurotrophic factor such as bone-derived neurotrophic factor (BDNF), neurotrophin-3, -4, -5, or -6 (NT-3, NT-4, NT-5, or NT-6), or a nerve growth factor such as NGF-b; transforming growth factor (TGF) such as TGF-alpha and TGF-beta, including TGF-β1, TGF-β2, TGF-β3, TGF-β4, or TGF-β5; insulin-like growth factor-I and -II (IGF-I and IGF-II); des(1-3)-IGF-I (brain IGF-I), insulin-like growth factor binding proteins (IGFBPs); CD proteins such as CD3, CD4, CD8, CD19 and CD20; erythropoietin; osteoinductive factors; immunotoxins; a bone morphogenetic protein (BMP); a chemokine such as CXCL12 and CXCR4; an interferon such as interferon-alpha, -beta, and -gamma; colony stimulating factors (CSFs), e.g., M-CSF, GM-CSF, and G-CSF; a cytokine such as interleukins (ILs), e.g., IL-1 to IL-10; midkine; superoxide dismutase; T-cell receptors; surface membrane proteins; decay accelerating factor; viral antigen such as, for example, a portion of the AIDS envelope; transport proteins; homing receptors; addressins; regulatory proteins; integrins such as CD11a, CD11b, CD11c, CD18, an ICAM, VLA-4 and VCAM; ephrins; Bv8; Delta-like ligand 4 (DLL4); Del-1; BMP9; BMP10; Follistatin; Hepatocyte growth factor (HGF)/scatter factor (SF); Alk1; Robo4; ESM1; Perlecan; EGF-like domain, multiple 7 (EGFL7); CTGF and members of its family; thrombospondins such as thrombospondinl and thrombospondin2; collagens such as collagen IV and collagen XVIII; neuropilins such as NRP1 and NRP2; Pleiotrophin (PTN); Progranulin; Proliferin; Notch proteins such as Notch1 and Notch4; semaphorins such as Sema3A, Sema3C, and Sema3F; a tumor associated antigen such as CA125 (ovarian cancer antigen); immunoadhesins; and fragments and/or variants of any of the above-listed polypeptides as well as antibodies, including antibody fragments, binding to one or more protein, including, for example, any of the above-listed proteins.

The term “antibody” herein is used in the broadest sense and specifically covers monoclonal antibodies (including full length monoclonal antibodies), polyclonal antibodies, multispecific antibodies (e.g., bispecific, trispecific, etc.), and antibody fragments so long as they exhibit the desired biological activity.

An “isolated” polypeptide (e.g., an isolated antibody) is one which has been identified and separated and/or recovered from a component of its natural environment. Contaminant components of its natural environment are materials which would interfere with research, diagnostic or therapeutic uses for the polypeptide, and may include enzymes, hormones, and other proteinaceous or nonproteinaceous solutes. Isolated polypeptide includes the polypeptide in situ within recombinant cells since at least one component of the polypeptide's natural environment will not be present. Ordinarily, however, isolated polypeptide will be prepared by at least one purification step.

“Native antibodies” are usually heterotetrameric glycoproteins of about 150,000 Daltons, composed of two identical light (L) chains and two identical heavy (H) chains. Each light chain is linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (V_{H}) followed by a number of constant domains. Each light chain has a variable domain at one end (V_{L}) and a constant domain at its other end; the constant domain of the light chain is aligned with the first constant domain of the heavy chain, and the light chain variable domain is aligned with the variable domain of the heavy chain. Particular amino acid residues are believed to form an interface between the light chain and heavy chain variable domains.

The term “constant domain” refers to the portion of an immunoglobulin molecule having a more conserved amino acid sequence relative to the other portion of the immunoglobulin, the variable domain, which contains the antigen binding site. The constant domain contains the C_{H}1, C_{H}2 and C_{H}3 domains (collectively, CH) of the heavy chain and the CHL (or CL) domain of the light chain.

The “variable region” or “variable domain” of an antibody refers to the amino-terminal domains of the heavy or light chain of the antibody. The variable domain of the heavy chain may be referred to as “V_{H}.” The variable domain of the light chain may be referred to as “V_{L}.” These domains are generally the most variable parts of an antibody and contain the antigen-binding sites.

The term “variable” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called hypervariable regions (HVRs) both in the light-chain and the heavy-chain variable domains. The more highly conserved portions of variable domains are called the framework regions (FR). The variable domains of native heavy and light chains each comprise four FR regions, largely adopting a beta-sheet configuration, connected by three HVRs, which form loops connecting, and in some cases forming part of, the beta-sheet structure. The HVRs in each chain are held together in close proximity by the FR regions and, with the HVRs from the other chain, contribute to the formation of the antigen-binding site of antibodies (see Kabat et al., Sequences of Proteins of Immunological Interest, Fifth Edition, National Institute of Health, Bethesda. Md. (1991)). The constant domains are not involved directly in the binding of an antibody to an antigen, but exhibit various effector functions, such as participation of the antibody in antibody-dependent cellular toxicity.

The “light chains” of antibodies (immunoglobulins) from any mammalian species can be assigned to one of two clearly distinct types, called kappa (“κ”) and lambda (“λ”), based on the amino acid sequences of their constant domains.

The term IgG “isotype” or “subclass” as used herein is meant any of the subclasses of immunoglobulins defined by the chemical and antigenic characteristics of their constant regions. Depending on the amino acid sequences of the constant domains of their heavy chains, antibodies (immunoglobulins) can be assigned to different classes. There are five major classes of immunoglobulins: IgA, IgD, IgE, IgG, and IgM, and several of these may be further divided into subclasses (isotypes), e.g., IgG₁, IgG₂, IgG₃, IgG₄, IgA₁, and IgA₂. The heavy chain constant domains that correspond to the different classes of immunoglobulins are called α, γ, ε, γ, and μ, respectively. The subunit structures and three-dimensional configurations of different classes of immunoglobulins are well known and described generally in, for example, Abbas et al. Cellular and Mol. Immunology, 4th ed., W. B. Saunders, Co., 2000. An antibody may be part of a larger fusion molecule, formed by covalent or non-covalent association of the antibody with one or more other proteins or peptides.

The terms “full length antibody,” “intact antibody”, and “whole antibody” are used herein interchangeably to refer to an antibody in its substantially intact form, not antibody fragments as defined below. The terms particularly refer to an antibody with heavy chains that contain an Fc region.

“Antibody fragments” comprise a portion of an intact antibody, preferably comprising the antigen binding region thereof. Examples of antibody fragments include Fab, Fab′, F(ab′)2, and Fv fragments; diabodies; linear antibodies; single-chain antibody molecules; and multispecific antibodies formed from antibody fragments.

Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)2 fragment that has two antigen-combining sites and is still capable of cross-linking antigen. The Fab fragment contains the heavy- and light-chain variable domains and also contains the constant domain of the light chain and the first constant domain (CH1) of the heavy chain. Fab′ fragments differ from Fab fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

“Fv” is the minimum antibody fragment which contains a complete antigen-binding site. In one embodiment, a two-chain Fv species consists of a dimer of one heavy- and one light-chain variable domain in tight, non-covalent association. In a single-chain Fv (scFv) species, one heavy- and one light-chain variable domain can be covalently linked by a flexible peptide linker such that the light and heavy chains can associate in a “dimeric” structure analogous to that in a two-chain Fv species. It is in this configuration that the three HVRs of each variable domain interact to define an antigen-binding site on the surface of the VH-VL dimer. Collectively, the six HVRs confer antigen-binding specificity to the antibody. However, even a single variable domain (or half of an Fv comprising only three HVRs specific for an antigen) has the ability to recognize and bind antigen, although at a lower affinity than the entire binding site.

“Single-chain Fv” or “scFv” antibody fragments comprise the VH and VL domains of antibody, wherein these domains are present in a single polypeptide chain. Generally, the scFv polypeptide further comprises a polypeptide linker between the VH and VL domains which enables the scFv to form the desired structure for antigen binding. For a review of scFv, see, e.g., Pluckthiin, in The Pharmacology of Monoclonal Antibodies, vol. 113, Rosenburg and Moore eds., Springer-Verlag, New York, pp. 269-315, 1994.

The term “diabodies” refers to antibody fragments with two antigen-binding sites, which fragments comprise a heavy-chain variable domain (VH) connected to a light-chain variable domain (VL) in the same polypeptide chain (VH-VL). By using a linker that is too short to allow pairing between the two domains on the same chain, the domains are forced to pair with the complementary domains of another chain and create two antigen-binding sites. Diabodies may be bivalent or bispecific. Diabodies are described more fully in, for example, EP 404,097; WO 1993/01161; Hudson et al., Nat. Med. 9:129-134 (2003); and Hollinger et al., Proc. Natl. Acad. Sci. USA 90: 6444-6448 (1993). Triabodies and tetrabodies are also described in Hudson et al., Nat. Med. 9:129-134 (2003).

The term “monoclonal antibody” as used herein refers to an antibody obtained from a population of substantially homogeneous antibodies, e.g., the individual antibodies comprising the population are identical except for possible mutations, e.g., naturally occurring mutations, that may be present in minor amounts. Thus, the modifier “monoclonal” indicates the character of the antibody as not being a mixture of discrete antibodies. In some embodiments, such a monoclonal antibody typically includes an antibody comprising a polypeptide sequence that binds a target, wherein the target-binding polypeptide sequence was obtained by a process that includes the selection of a single target binding polypeptide sequence from a plurality of polypeptide sequences. For example, the selection process can be the selection of a unique clone from a plurality of clones, such as a pool of hybridoma clones, phage clones, or recombinant DNA clones. It should be understood that a selected target binding sequence can be further altered, for example, to improve affinity for the target, to humanize the target binding sequence, to improve its production in cell culture, to reduce its immunogenicity in vivo, to create a multispecific antibody, etc., and that an antibody comprising the altered target binding sequence is also a monoclonal antibody of the present disclosure. In contrast to polyclonal antibody preparations, which typically include different antibodies directed against different determinants (epitopes), each monoclonal antibody of a monoclonal antibody preparation is directed against a single determinant on an antigen. In addition to their specificity, monoclonal antibody preparations are advantageous in that they are typically uncontaminated by other immunoglobulins.

The modifier “monoclonal” indicates the character of the antibody as being obtained from a substantially homogeneous population of antibodies, and is not to be construed as requiring production of the antibody by any particular method. For example, the monoclonal antibodies to be used in accordance with the present disclosure may be made by a variety of techniques, including, for example, the hybridoma method (e.g., Kohler and Milstein, Nature, 256:495-97 (1975); Hongo et al., Hybridoma, 14 (3): 253-260 (1995), Harlow et al., Antibodies: A Laboratory Manual, (Cold Spring Harbor Laboratory Press, 2nd ed. 1988); Hammerling et al., in: Monoclonal Antibodies and T-Cell Hybridomas 563-681 (Elsevier, N.Y., 1981)), recombinant DNA methods (see, e.g., U.S. Pat. No. 4,816,567), phage-display technologies (see, e.g., Clackson et al., Nature, 352: 624-628 (1991); Marks et al., J. Mol. Biol. 222: 581-597 (1992); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132 (2004), and technologies for producing human or human-like antibodies in animals that have parts or all of the human immunoglobulin loci or genes encoding human immunoglobulin sequences (see, e.g., WO 1998/24893; WO 1996/34096; WO 1996/33735; WO 1991/10741; Jakobovits et al., Proc. Natl. Acad. Sci. USA 90: 2551 (1993); Jakobovits et al., Nature 362: 255-258 (1993); Bruggemann et al., Year in Immunol. 7:33 (1993); U.S. Pat. Nos. 5,545,807; 5,545,806; 5,569,825; 5,625,126; 5,633,425; and 5,661,016; Marks et al., Bio/Technology 10: 779-783 (1992); Lonberg et al., Nature 368: 856-859 (1994); Morrison, Nature 368: 812-813 (1994); Fishwild et al., Nature Biotechnol. 14: 845-851 (1996); Neuberger, Nature Biotechnol. 14: 826 (1996); and Lonberg and Huszar, Intern. Rev. Immunol. 13: 65-93 (1995).

The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (see, e.g., U.S. Pat. No. 4,816,567; and Morrison et al., Proc. Natl. Acad. Sci. USA 81:6851-6855 (1984)). Chimeric antibodies include PRIMATTZED® antibodies wherein the antigen-binding region of the antibody is derived from an antibody produced by, e.g., immunizing macaque monkeys with the antigen of interest.

“Humanized” forms of non-human (e.g., murine) antibodies are chimeric antibodies that contain minimal sequence derived from non-human immunoglobulin. In one embodiment, a humanized antibody is a human immunoglobulin (recipient antibody) in which residues from a HVR of the recipient are replaced by residues from a HVR of a non-human species (donor antibody) such as mouse, rat, rabbit, or nonhuman primate having the desired specificity, affinity, and/or capacity. In some instances, FR residues of the human immunoglobulin are replaced by corresponding non-human residues. Furthermore, humanized antibodies may comprise residues that are not found in the recipient antibody or in the donor antibody. These modifications may be made to further refine antibody performance. In general, a humanized antibody will comprise substantially all of at least one, and typically two, variable domains, in which all or substantially all of the hypervariable loops correspond to those of a non-human immunoglobulin, and all or substantially all of the FRs are those of a human immunoglobulin sequence. The humanized antibody optionally will also comprise at least a portion of an immunoglobulin constant region (Fc), typically that of a human immunoglobulin. For further details, see, e.g., Jones et al., Nature 321:522-525 (1986); Riechmann et al., Nature 332:323-329 (1988); and Presta, Curr. Op. Struct. Biol. 2:593-596 (1992). See also, e.g., Vaswani and Hamilton, Ann. Allergy, Asthma & Immunol. 1:105-115 (1998); Harris, Biochem. Soc. Transactions 23:1035-1038 (1995); Hurle and Gross, Cure. Op. Biotech. 5:428-433 (1994); and U.S. Pat. Nos. 6,982,321 and 7,087,409.

A “human antibody” is one which possesses an amino acid sequence which corresponds to that of an antibody produced by a human and/or has been made using any of the techniques for making human antibodies as disclosed herein. This definition of a human antibody specifically excludes a humanized antibody comprising non-human antigen-binding residues. Human antibodies can be produced using various techniques known in the art, including phage-display libraries. Hoogenboom and Winter, J. Mol. Biol., 227:381 (1991); Marks et al., J. Mol. Biol., 222:581 (1991). Also available for the preparation of human monoclonal antibodies are methods described in Cole et al., Monoclonal Antibodies and Cancer Therapy, Alan R. Liss, p. 77 (1985); Boemer et al., J. Immunol., 147(1):86-95 (1991). See also van Dijk and van de Winkel, Curr. Opin. Pharmacol., 5: 368-74 (2001). Human antibodies can be prepared by administering the antigen to a transgenic animal that has been modified to produce such antibodies in response to antigenic challenge, but whose endogenous loci have been disabled, e.g., immunized xenomice (see, e.g., U.S. Pat. Nos. 6,075,181 and 6,150,584 regarding XENOMOUSE™ technology). See also, for example, Li et al., Proc. Natl. Acad. Sci. USA, 103:3557-3562 (2006) regarding human antibodies generated via a human B-cell hybridoma technology.

The term “hypervariable region,” “HVR,” or “HV,” when used herein refers to the regions of an antibody variable domain which are hypervariable in sequence and/or form structurally defined loops. Generally, antibodies comprise six HVRs; three in the VH (H1, H2, H3), and three in the VL (L1, L2, L3). In native antibodies, H3 and L3 display the most diversity of the six HVRs, and H3 in particular is believed to play a unique role in conferring fine specificity to antibodies. See, e.g., Xu et al., Immunity 13:37-45 (2000); Johnson and Wu, in Methods in Molecular Biology 248:1-25 (Lo, ed., Human Press, Totowa, N.J., 2003). Indeed, naturally occurring camelid antibodies consisting of a heavy chain only are functional and stable in the absence of light chain. See, e.g., Hamers-Casterman et al., Nature 363:446-448 (1993); Sheriff et al., Nature Siruct. Biol. 3:733-736 (1996). In some embodiments, the HVRs are Complementarity Determining Regions (CDRs).

A number of HVR delineations are in use and are encompassed herein. The Kabat Complementarity Determining Regions (CDRs) are based on sequence variability and are the most commonly used (Kabat et al., Sequences of Proteins of immunological Interest, 5th Ed. Public Health Service, National Institutes of Health, Bethesda, Md. (1991)). Chothia refers instead to the location of the structural loops (Chothia and Leskm J. Mol. Biol. 196:901-917 (1987)). The AbM HVRs represent a compromise between the Kabat HVRs and Chothia structural loops, and are used by Oxford Molecular's AbM antibody modeling software. The “contact” HVRs are based on an analysis of the available complex crystal structures. The residues from each of these HVRs are noted below.

| Loop | Kabat | AbM | Chothia | Contact |
|---|---|---|---|---|
| L1 | L24-L34 | L24-L34 | L26-L32 | L30-L36 |
| L2 | L50-L56 | L50-L56 | L50-L52 | L46-L55 |
| L3 | L89-L97 | L89-L97 | L91-L96 | L89-L96 |
| H1 | H31-H35B | H26-H35B | H26-H32 | H30-H35B |
| | | | | (Kabat Numbering) |
| H1 | H31-H35 | H26-H35 | H26-H32 | H30-H35 |
| | | | | (Chothia Numbering) |
| H2 | H50-H65 | H50-H58 | H53-H55 | H47-H58 |
| H3 | H95-H102 | H95-H102 | H96-H101 | H93-H101 |

HVRs may comprise “extended HVRs” as follows: 24-36 or 24-34 (L1), 46-56 or 50-56 (L2) and 89-97 or 89-96 (L3) in the VL and 26-35 (H1), 50-65 or 49-65 (H2) and 93-102, 94-102, or 95-102 (H3) in the VH. The variable domain residues are numbered according to Kabat et al., supra, for each of these definitions.

“Framework” or “FR” residues are those variable domain residues other than the HVR residues as herein defined.

The terms “variable domain residue numbering as in Kabat”; “amino acid position numbering as in Kabat”, “residue numbering is according to Kabat numbering”, and variations thereof, refers to the numbering system used for heavy chain variable domains or light chain variable domains of the compilation of antibodies in Kabat et al., supra. Using this numbering system, the actual linear amino acid sequence may contain fewer or additional amino acids corresponding to a shortening of, or insertion into, a FR or HVR of the variable domain. For example, a heavy chain variable domain may include a single amino acid insert (residue 52a according to Kabat) after residue 52 of H2 and inserted residues (e.g. residues 82a, 82b, and 82c, etc. according to Kabat) after heavy chain FR residue 82. The Kabat numbering of residues may be determined for a given antibody by alignment at regions of homology of the sequence of the antibody with a “standard” Kabat numbered sequence

The Kabat numbering system is generally used when referring to a residue in the variable domain (approximately residues 1-107 of the light chain and residues 1-113 of the heavy chain) (e.g., Kabat et al., Sequences of Immunological Interest. 5th Ed. Public Health Service, National Institutes of Health, Bethesda, Md. (1991)). The terms “EU numbering system”, “EU index”, “residue numbering is according to EU numbering”, and variations thereof, are generally used when referring to a residue in an immunoglobulin heavy chain constant region (e.g., the EU index reported in Kabat et al., supra). The “EU index as in Kabat” refers to the residue numbering of the human IgG1 EU antibody.

The term “multispecific antibody” is used in the broadest sense and specifically covers an antibody comprising an antigen-binding domain that has polyepitopic specificity (i.e., is capable of specifically binding to two, or more, different epitopes on one biological molecule or is capable of specifically binding to epitopes on two, or more, different biological molecules). In some embodiments, an antigen-binding domain of a multispecific antibody (such as a bispecific antibody) comprises two VH/VL units, wherein a first VH/VL unit specifically binds to a first epitope and a second VH/VL unit specifically binds to a second epitope, wherein each VH/VL unit comprises a heavy chain variable domain (VH) and a light chain variable domain (VL). Such multispecific antibodies include, but are not limited to, full length antibodies, antibodies having two or more VL and VH domains, antibody fragments such as Fab, Fv, dsFv, scFv, diabodies, bispecific diabodies and triabodies, antibody fragments that have been linked covalently or non-covalently. A VH/VL unit that further comprises at least a portion of a heavy chain constant region and/or at least a portion of a light chain constant region may also be referred to as a “hemimer” or “half antibody.” In some embodiments, a half antibody comprises at least a portion of a single heavy chain variable region and at least a portion of a single light chain variable region. In some such embodiments, a bispecific antibody that comprises two half antibodies and binds to two antigens comprises a first half antibody that binds to the first antigen or first epitope but not to the second antigen or second epitope and a second half antibody that binds to the second antigen or second epitope and not to the first antigen or first epitope. According to some embodiments, the multispecific antibody is an IgG antibody that binds to each antigen or epitope with an affinity of 5 M to 0.001 pM, 3 M to 0.001 pM, 1 M to 0.001 pM, 0.5 M to 0.001 pM, or 0.1 M to 0.001 pM. In some embodiments, a hemimer comprises a sufficient portion of a heavy chain variable region to allow intramolecular disulfide bonds to be formed with a second hemimer. In some embodiments, a hemimer comprises a knob mutation or a hole mutation, for example, to allow heterodimerization with a second hemimer or half antibody that comprises a complementary hole mutation or knob mutation. Knob mutations and hole mutations are discussed further below.

A “bispecific antibody” is a multispecific antibody comprising an antigen-binding domain that is capable of specifically binding to two different epitopes on one biological molecule or is capable of specifically binding to epitopes on two different biological molecules. A bispecific antibody may also be referred to herein as having “dual specificity” or as being “dual specific.” Unless otherwise indicated, the order in which the antigens bound by a bispecific antibody are listed in a bispecific antibody name is arbitrary. In some embodiments, a bispecific antibody comprises two half antibodies, wherein each half antibody comprises a single heavy chain variable region and optionally at least a portion of a heavy chain constant region, and a single light chain variable region and optionally at least a portion of a light chain constant region. In some embodiments, a bispecific antibody comprises two half antibodies, wherein each half antibody comprises a single heavy chain variable region and a single light chain variable region and does not comprise more than one single heavy chain variable region and does not comprise more than one single light chain variable region. In some embodiments, a bispecific antibody comprises two half antibodies, wherein each half antibody comprises a single heavy chain variable region and a single light chain variable region, and wherein the first half antibody binds to a first antigen and not to a second antigen and the second half antibody binds to the second antigen and not to the first antigen.

The term “knob-into-hole” or “KnH” technology as used herein refers to the technology directing the pairing of two polypeptides together in vitro or in vivo by introducing a protuberance (knob) into one polypeptide and a cavity (hole) into the other polypeptide at an interface in which they interact. For example, KnHs have been introduced in the Fc:Fc binding interfaces, CL:CH1 interfaces or VH/VL interfaces of antibodies (see. e.g., US 2011/0287009, US2007/0178552, WO 96/027011, WO 98/050431, and Zhu et al., 1997, Protein Science 6:781-788). In some embodiments, KnHs drive the pairing of two different heavy chains together during the manufacture of multispecific antibodies. For example, multispecific antibodies having KnH in their Fc regions can further comprise single variable domains linked to each Fc region, or further comprise different heavy chain variable domains that pair with similar or different light chain variable domains. KnH technology can also be used to pair two different receptor extracellular domains together or any other polypeptide sequences that comprises different target recognition sequences (e.g., including affibodies, peptibodies and other Fc fusions).

The term “knob mutation” as used herein refers to a mutation that introduces a protuberance (knob) into a polypeptide at an interface in which the polypeptide interacts with another polypeptide. In some embodiments, the other polypeptide has a hole mutation (see e.g., U.S. Pat. Nos. 5,731,168, 5,807,706, 5,821,333, 7,695,936, 8,216,805, each incorporated herein by reference in its entirety).

The term “hole mutation” as used herein refers to a mutation that introduces a cavity (hole) into a polypeptide at an interface in which the polypeptide interacts with another polypeptide. In some embodiments, the other polypeptide has a knob mutation (see e.g., U.S. Pat. Nos. 5,731,168, 5,807,706, 5,821,333, 7,695,936, 8,216,805, each incorporated herein by reference in its entirety).

The expression “linear antibodies” refers to the antibodies described in Zapata et al. (1995 Protein Eng, 8(10):1057-1062). Briefly, these antibodies comprise a pair of tandem Fd segments (VH-CH1-VH-CH1) which; together with complementary light chain polypeptides, form a pair of antigen binding regions. Linear antibodies can be bispecific or monospecific.

### II. Polypeptide Formulations and Preparation

Certain aspects of the present disclosure relate to formulations comprising a polypeptide, N-acetyl-DL-tryptophan (NAT), and L-methionine, wherein the NAT and L-methionine reduce or prevent oxidation of the polypeptide. In some embodiments, the polypeptide is susceptible to oxidation. In some embodiments, methionine, cysteine, histidine, tryptophan, and/or tyrosine residues in the polypeptide are susceptible to oxidation. In some embodiments, one or more tryptophan residues in the polypeptide are susceptible to oxidation. In some embodiments, one or more methionine residues in the polypeptide are susceptible to oxidation. In some embodiments, one or more tryptophan and one or more methionine residues in the polypeptide are susceptible to oxidation. In some embodiments, the polypeptide is antibody. In some embodiments, the formulation further comprises at least one additional polypeptide according to any of the polypeptides described herein. In some embodiments, the formulation further comprises one or more excipients. In some embodiments, the formulation is a liquid formulation. In some embodiments, the formulation is an aqueous formulation. In some embodiments, the formulation is a pharmaceutical formulation (e.g., suitable for administration to a human subject).

In some embodiments, the concentration of NAT in the formulation is from about 0.01 mM to about 25 mM (such as about any of 0.01, 0.025, 0.05, 0.075, 0.1, 0.2, 0.3, 0.4, 0.5, 0.6, 0.7, 0.8, 0.9, 1.0, 2.0, 3.0, 4.0, 5.0, 6.0, 7.0, 8.0, 9.0, 10.0, 11.0, 12.0, 13.0, 14.0, 15.0, 16.0, 17.0, 18.0, 19.0, 20.0, 21.0, 22.0, 23.0, 24.0, or 25.0 mM, including any ranges between these values), or up to the highest concentration that the NAT is soluble in the formulation. In some embodiments, the concentration of NAT in the formulation is from about 0.05 to about 1 mM. In some embodiments, the concentration of NAT in the formulation is from about 0.05 to about 0.3 mM. In some embodiments, the concentration of NAT in the formulation is about 0.05 mM. In some embodiments, the concentration of NAT in the formulation is about 0.1 mM. In some embodiments, the concentration of NAT in the formulation is about 0.3 mM. In some embodiments, the concentration of NAT in the formulation is about 1.0 mM. In some embodiments, the concentration of NAT in the formulation is about 1 mM.

In some embodiments, the NAT reduces or prevents oxidation of one or more tryptophan residues in the polypeptide. In some embodiments, the NAT reduces or prevents oxidation of one or more tryptophan residues in the polypeptide by a reactive oxygen species (ROS). In some embodiments, the reactive oxygen species is selected from a singlet oxygen, a superoxide (O₂—), an alkoxyl radical, a peroxyl radical, a hydrogen peroxide (H₂O₂), a dihydrogen trioxide (H₂O₃), a hydrotrioxy radical (HO₃.), ozone (O₃), a hydroxyl radical, and/or an alkyl peroxide.

In some embodiments, the polypeptide is an antibody, and the NAT reduces or prevents oxidation of one or more tryptophan residues in the antibody. In some embodiments, the one or more tryptophan residues are located within the light chain constant region and/or the heavy chain constant region of the antibody. In some embodiments, the one or more tryptophan residues are located within the light chain variable region (e.g., an HVR-L1, HVR-L2, and/or HVR-L3) and/or the heavy chain variable region (e.g., an HVR-H1, HVR-H2, and/or HVR-H3) of the antibody. In some embodiments, the one or more tryptophan residues are located in the heavy chain variable region of an antibody. In some embodiments, the one or more tryptophan residues are located in a framework region of the heavy chain variable region. In some embodiments, the one or more tryptophan residues comprises W103 (according to Kabat numbering). In some embodiments, the one or more tryptophan residues are located in an HVR-H1, HVR-H2, and/or HVR-H3 of the antibody (e.g., an HVR-H1 and/or HVR-H3). In some embodiments, the one or more tryptophan residues comprises W33, W36, W52, W52a, W99, W100a, W100b and/or W103 (according to Kabat numbering). In some embodiments, the one or more tryptophan residues comprises W33 and/or W36, W99 and/or W100a. In some embodiments, inclusion of NAT in a formulation of the present disclosure reduces or prevents oxidation of the antibody at residues W33, W36, W52a, WW99, W100a, W110b, and/or W103 (e.g., as compared to one or more corresponding tryptophan residue(s) in the polypeptide in a liquid formulation lacking NAT). In some embodiments, the one or more tryptophan residues are located in an HVR-L1, HVR-L2, and/or HVR-L3 of the antibody. In some embodiments, the one or more tryptophan residues comprises W94, W31 and/or W91.

In some embodiments, the concentration of L-methionine in the formulation is from about 1.0 mM to about 125.0 mM (such as about any of 1.0, 2.0, 3.0, 4.0, 5.0, 6.0, 7.0, 8.0, 9.0, 10.0, 15.0, 20.0, 25.0, 30.0, 35.0, 40.0, 45.0, 50.0, 55.0, 60.0, 65.0, 70.0, 75.0, 80.0, 85.0, 90.0, 95.0, 100.0, 105.0, 110.0, 115.0, 120.0, or 125.0 mM, including any ranges between these values), or up to the highest concentration that the L-methionine is soluble in the formulation. In some embodiments, the concentration of L-methionine in the formulation is from about 5.0 to about 25.0 mM. In some embodiments, the concentration of L-methionine in the formulation is about 5.0 mM.

In some embodiments, the L-methionine reduces or prevents oxidation of one or more methionine residues in the polypeptide. In some embodiments, the L-methionine reduces or prevents oxidation of one or more methionine residues in the polypeptide by a reactive oxygen species (ROS). In some embodiments, the reactive oxygen species is selected from a singlet oxygen, a superoxide (O₂—), an alkoxyl radical, a peroxyl radical, a hydrogen peroxide (H₂O₂), a dihydrogen trioxide (H₂O₃), a hydrotrioxy radical (HO₃.), ozone (O₃), a hydroxyl radical, and/or an alkyl peroxide.

In some embodiments, the polypeptide is an antibody, and the L-methionine reduces or prevents oxidation of one or more methionine residues in the antibody. In some embodiments, the one or more methionine residues are located within the light chain variable region (e.g., an HVR-L1, HVR-L2, and/or HVR-L3) and/or the heavy chain variable region (e.g., an HVR-H1, HVR-H2, and/or and HVR-H3) of the antibody. In some embodiments, the one or more methionine residues are located in the heavy chain variable region of an antibody. In some embodiments, the one or more methionine residues are located in a framework region of the heavy chain variable region. In some embodiments, the one or more methionine residues comprises M82 (according to Kabat numbering). In some embodiments, the one or more tryptophan residues are located in an HVR-H1, HVR-H2, and/or HVR-H3 of the antibody (e.g., an HVR-H1). In some embodiments, the one or more methionine residues comprises M34 (according to Kabat numbering). In some embodiments, the one or more methionine residues are located in an HVR-L1, HVR-L2, and/or HVR-L3 of the antibody (e.g., an HVR-L1). In some embodiments, the one or more methionine residues are located in the light chain; e.g., at sites M30, M33, M92. In some embodiments, the one or more methionine residues are located in the heavy chain; e.g., at sites M82, M99, M57, M58, M62, M64 and other sites between 95-102. In some embodiments, the one or more methionine residues are located within the light chain constant region and/or the heavy chain constant region of the antibody. In some embodiments, the one or more methionine residues are located in the heavy chain constant region of an antibody (e.g., an IgG1 antibody). In some embodiments, the one or more methionine residues comprises M252, M35 and/or M428 (according to EU numbering). In some embodiments, inclusion of L-methionine in a formulation of the present disclosure reduces or prevents oxidation of the antibody at residues M34, M82, M252, and/or M428 (e.g., as compared to one or more corresponding methionine residue(s) in the polypeptide in a liquid formulation lacking L-methionine).

In some embodiments, inclusion of NAT in a formulation of the present disclosure increases oxidation of the antibody at one or more methionine residues (e.g., any of the methionine residues described above, such as an Fc region methionine at position M252 and/or M428). In some embodiments, inclusion of L-methionine in the formulation reduces or prevents NAT-induced and/or amplified oxidation of one or more methionine residues in the antibody (e.g., any of the methionine residues described above, such as an Fc region methionine at position M252, M358 and/or M428). In some embodiments, a liquid formulation of the present disclosure comprises NAT at any of the concentrations described herein and L-methionine at any of the concentrations described herein. In some embodiments, the liquid formulation comprises Nat at a concentration of about 0.3 mM and L-methionine at a concentration of about 5.0 mM. In some embodiments, the liquid formulation comprises NAT at a concentration of about 1.0 mM and L-methionine at a concentration of about 5.0 mM.

In some embodiments, liquid formulations provided by the present disclosure comprise a polypeptide, NAT, and L-methionine (where the NAT and L-methionine reduce or prevent oxidation of the polypeptide in the liquid formulation), wherein the oxidation of the polypeptide (e.g., the oxidation of one or more tryptophan residues and/or one or more methionine residues in the polypeptide) is reduced by about 40% to about 100% (e.g., as compared to one or more corresponding tryptophan residues and/or one or more corresponding methionine residues in the polypeptide in a liquid formulation lacking NAT and/or L-methionine). In some embodiments, the oxidation of the polypeptide (e.g., the oxidation of one or more tryptophan residues and/or one or more methionine residues in the polypeptide) is reduced by about any of 40%, 45%, 50%, 55%, 60%, 65%, 70%, 75%, 80%, 85%, 90%, 95%, 96%, 97%, 98%, 99%, or 100%, including any ranges between these values (e.g., as compared to one or more corresponding tryptophan residues and/or one or more corresponding methionine residues in the polypeptide in a liquid formulation lacking NAT and/or L-methionine). Any suitable method of measuring polypeptide oxidation known in the art may be used, including, for example, the methods described in Example 1 below (and the references cited therein).

The amount of oxidation in a polypeptide can be determined, for example, using one or more of RP-HPLC, LC/MS, or tryptic peptide mapping. In some embodiments, the oxidation in a polypeptide is determined as a percentage using one or more of RP-HPLC, LC/MS, or tryptic peptide mapping and the formula of:

$$\% Oxidation = 100 \times \frac{Oxidized Peak Area}{Peak Area + Oxidized Peak Area}$$

In some embodiments, liquid formulations provided by the present disclosure comprise a polypeptide, NAT, and L-methionine (where the NAT and L-methionine reduce or prevent oxidation of the polypeptide in the liquid formulation), wherein no more than about 40% to about 0% of the polypeptide is oxidized (e.g., oxidized at one or more tryptophan residues and/or one or more methionine residues in the polypeptide). In some embodiments, no more than about any of 40%, 35%, 30%, 25%, 20%, 15%, 10%, 5%, 4%, 3%, 2%, 1%, or 0%, including any ranges between these values, of the polypeptide is oxidized (e.g., oxidized at one or more tryptophan residues and/or one or more methionine residues in the polypeptide).

In some embodiments, liquid formulations provided by the present disclosure comprise a polypeptide, NAT, and L-methionine (where the NAT and L-methionine reduce or prevent oxidation of the polypeptide in the liquid formulation), wherein the oxidation of at least one oxidation labile tryptophan residue (e.g., any one or more of the tryptophan residues of an antibody as described herein) in the polypeptide is reduced by about 40% to about 100% (e.g., as compared to one or more corresponding tryptophan residue(s) in the polypeptide in a formulation lacking NAT). In some embodiments, the oxidation of the oxidation labile tryptophan residue(s) in the polypeptide is reduced by about any of 40%, 45%, 50%, 55%, 60%, 65%, 70%, 75%, 80%, 85%, 90%, 95%, 96%, 97%, 98%, 99%, or 100%, including any ranges between these values. In some embodiments, the oxidation of each of the oxidation labile tryptophan residues in the polypeptide is reduced by about 40% to about 100% (such as about any of 40%, 45%, 50%, 55%, 60%, 65%, 70%, 75%, 80%, 85%, 90%, 95%, 96%, 97%, 98%, 99%, or 100%, including any ranges between these values).

In some embodiments, liquid formulations provided by the present disclosure comprise a polypeptide, NAT, and L-methionine (where the NAT and L-methionine reduce or prevent oxidation of the polypeptide in the liquid formulation), wherein no more than about 40% to about 0% of at least one oxidation labile tryptophan residue (e.g., any one or more of the tryptophan residues of an antibody as described herein) in the polypeptide is oxidized. In some embodiments, no more than about any of 40%, 35%, 30%, 25%, 20%, 15%, 10%, 5%, 4%, 3%, 2%, 1%, or 0%, including any ranges between these values, of the oxidation labile tryptophan residue(s) in the polypeptide is oxidized. In some embodiments, no more than about 40% to about 0% (such as no more than about any of 40%, 35%, 30%, 25%, 20%, 15%, 10%, 5%, 4%, 3%, 2%, 1%, or 0%, including any ranges between these values) of each of the oxidation labile tryptophan residues in the polypeptide is oxidized.

In some embodiments, liquid formulations provided by the present disclosure comprise a polypeptide, NAT, and L-methionine (where the NAT and L-methionine reduce or prevent oxidation of the polypeptide in the liquid formulation), wherein the oxidation of at least one oxidation labile methionine residue (e.g., any one or more of the methionine residues of an antibody as described herein) in the polypeptide is reduced by about 40% to about 100% (e.g., as compared to one or more corresponding methionine residue(s) in the polypeptide in a formulation lacking L-methionine). In some embodiments, the oxidation of the oxidation labile methionine residue(s) in the polypeptide is reduced by about any of 40%, 45%, 50%, 55%, 60%, 65%, 70%, 75%, 80%, 85%, 90%, 95%, 96%, 97%, 98%, 99%, or 100%, including any ranges between these values. In some embodiments, the oxidation of each of the oxidation labile methionine residues in the polypeptide is reduced by about 40% to about 100% (such as about any of 40%, 45%, 50%, 55%, 60%, 65%, 70%, 75%, 80%, 85%, 90%, 95%, 96%, 97%, 98%, 99%, or 100%, including any ranges between these values).

In some embodiments, liquid formulations provided by the present disclosure comprise a polypeptide, NAT, and L-methionine (where the NAT and L-methionine reduce or prevent oxidation of the polypeptide in the liquid formulation), wherein no more than about 40% to about 0% of at least one oxidation labile methionine (e.g., any one or more of the methionine residues of an antibody as described herein) in the polypeptide is oxidized. In some embodiments, no more than about any of 40%, 35%, 30%, 25%, 20%, 15%, 10%, 5%, 4%, 3%, 2%, 1%, or 0%, including any ranges between these values, of the oxidation labile methionine residue in the polypeptide is oxidized. In some embodiments, no more than about 40% to about 0% (such as no more than about any of 40%, 35%, 30%, 25%, 20%, 15%, 10%, 5%, 4%, 3%, 2%, 1%, or 0%, including any ranges between these values) of each of the oxidation labile methionine residues in the polypeptide is oxidized.

In some embodiments, the polypeptide (e.g., the antibody) concentration in the formulation is about 1 mg/mL to about 250 mg/mL. In some embodiments, the polypeptide (e.g., the antibody) is a therapeutic polypeptide. Exemplary polypeptide concentrations in the formulation include from about 1 mg/mL to more than about 250 mg/mL, from about 1 mg/mL to about 250 mg/mL, from about 10 mg/mL to about 250 mg/mL, from about 15 mg/mL to about 225 mg/mL, from about 20 mg/mL to about 200 mg/mL, from about 25 mg/mL to about 175 mg/mL, from about 25 mg/mL to about 150 mg/mL, from about 25 mg/mL to about 100 mg/mL, from about 30 mg/mL to about 100 mg/mL or from about 45 mg/mL to about 55 mg/mL.

In some embodiments, the polypeptide is an antibody. In some embodiments, the antibody is a polyclonal antibody, a monoclonal antibody, a humanized antibody, a human antibody, a chimeric antibody, a multispecific antibody (e.g., bispecific, trispecific, etc.), or an antibody fragment. In some embodiments, the antibody is derived from an IgG1, IgG2, IgG3, or IgG4 antibody sequence. In some embodiments, the antibody is derived from an IgG1 antibody sequence.

In some embodiments, the formulation is aqueous. In some embodiments, the formulation further comprises one or more excipients. Any suitable excipient known in the art may be used in the formulations described herein, including, for example, a stabilizer, a buffer, a surfactant, a tonicity agent, and any combinations thereof. For example, a formulation of the present disclosure may comprise a monoclonal antibody, NAT as provided herein which prevents oxidation of the polypeptide (e.g., at one or more tryptophan residues), L-methionine as provided herein which prevents oxidation of the polypeptide (e.g., at one or more methionine residues) and a buffer that maintains the pH of the formulation to a desirable level. In some embodiments, a formulation provided herein has a pH of about 4.5 to about 9.0. In some embodiments, a formulation provided herein has a pH of about 4.5 to about 7.0. In some embodiments the pH is in the range from pH 4.0 to 8.5, in the range from pH 4.0 to 8.0, in the range from pH 4.0 to 7.5, in the range from pH 4.0 to 7.0, in the range from pH 4.0 to 6.5, in the range from pH 4.0 to 6.0, in the range from pH 4.0 to 5.5, in the range from pH 4.0 to 5.0, in the range from pH 4.0 to 4.5, in the range from pH 4.5 to 9.0, in the range from pH 5.0 to 9.0, in the range from pH 5.5 to 9.0, in the range from pH 6.0 to 9.0, in the range from pH 6.5 to 9.0, in the range from pH 7.0 to 9.0, in the range from pH 7.5 to 9.0, in the range from pH 8.0 to 9.0, in the range from pH 8.5 to 9.0, in the range from pH 5.7 to 6.8, in the range from pH 5.8 to 6.5, in the range from pH 5.9 to 6.5, in the range from pH 6.0 to 6.5, or in the range from pH 6.2 to 6.5. In some embodiments, the formulation has a pH of 6.2 or about 6.2. In some embodiments, the formulation has a pH of 6.0 or about 6.0. In some embodiments, the formulation further comprises at least one additional polypeptide according to any of the polypeptides described herein.

In some embodiments, the formulation provided herein is a pharmaceutical formulation suitable for administration to a subject. As used herein a “subject”, “patient”, or “individual” may refer to a human or a non-human animal. A “non-human animal” may refer to any animal not classified as a human, such as domestic, farm, or zoo animals, sports, pet animals (such as dogs, horses, cats, cows, etc.), as well as animals used in research. Research animals may refer without limitation to nematodes, arthropods, vertebrates, mammals, frogs, rodents (e.g., mice or rats), fish (e.g., zebrafish or pufferfish), birds (e.g., chickens), dogs, cats, and non-human primates (e.g., rhesus monkeys, cynomolgus monkeys, chimpanzees, etc.). In some embodiments, the subject, patient, or individual is a human.

Polypeptides and antibodies in the formulation may be prepared using any suitable method known in the art. An antibody (e.g., full length antibodies, antibody fragments and multispecific antibodies) in the formulation can be prepared using techniques available in the art, non-limiting exemplary methods of which are described in more detail in the following sections. The methods herein can be adapted by one of skill in the art for the preparation of formulations comprising other polypeptides such as peptide-based inhibitors. See Molecular Cloning: A Laboratory Manual (Sambrook et al., 4^{th} ed., Cold Spring Harbor Laboratory Press, Cold Spring Harbor, N.Y., 2012); Current Protocols in Molecular Biology (F. M. Ausubel, et al. eds., 2003); Short Protocols in Molecular Biology (Ausubel et al., eds., J. Wiley and Sons, 2002); Current Protocols in Protein Science, (Horswill et al., 2006); Antibodies, A Laboratory Manual (Harlow and Lane, eds., 1988); Culture of Animal Cells: A Manual of Basic Technique and Specialized Applications (R. I. Freshney, 6^{th} ed., J. Wiley and Sons, 2010) for generally well understood and commonly employed techniques and procedures for the production of therapeutic proteins, which are all incorporated herein by reference in their entirety.

In some embodiments, according to any of the formulations (e.g., liquid formulations) described herein, the formulation comprises two or more polypeptides (e.g., the formation is a co-formulation of two or more polypeptides). For example, in some embodiments, the formulation is a co-formulation comprising two or more polypeptides, NAT, and L-methionine, wherein the NAT and L-methionine reduce or prevent oxidation of at least one of the two or more polypeptides. In some embodiments, the NAT and L-methionine reduce or prevent oxidation of a plurality of the two or more polypeptides. In some embodiments, the NAT and L-methionine reduce or prevent oxidation of each of the two or more polypeptides. In some embodiments, at least one of the two or more polypeptides is an antibody, such as a polyclonal antibody, a monoclonal antibody, a humanized antibody, a human antibody, a chimeric antibody, a multispecific antibody, or an antibody fragment. In some embodiments, a plurality of the two or more polypeptides are antibodies, such as antibodies independently selected from among a polyclonal antibody, a monoclonal antibody, a humanized antibody, a human antibody, a chimeric antibody, a multispecific antibody, or an antibody fragment. In some embodiments, each of the two or more polypeptides is an antibody, such as an antibody independently selected from among a polyclonal antibody, a monoclonal antibody, a humanized antibody, a human antibody, a chimeric antibody, a multispecific antibody, or an antibody fragment. In some embodiments, one or more antibodies of the formulation are derived from an IgG1 antibody sequence. In some embodiments, the formulation is a liquid formulation. In some embodiments, the formulation is an aqueous formulation. In some embodiments, the formulation is a pharmaceutical formulation (e.g., suitable for administration to a human subject). In some embodiments, the pharmaceutical formulation is suitable for administration via any enteral route or parenteral route. The term “enteral route” of administration refers to the administration via any part of the gastrointestinal tract. Examples of enteral routes include oral, mucosal, buccal, and rectal route, or intragastric route. “Parenteral route” of administration refers to a route of administration other than enteral route. Examples of parenteral routes of administration include intravenous, intramuscular, intradermal, intraperitoneal, intratumor, intravesical, intraarterial, intrathecal, intracapsular, intraorbital, intravitreal, intracardiac, transtracheal, intraarticular, subcapsular, subarachnoid, intraspinal, epidural and intrasternal, subcutaneous, or topical administration. In some embodiments, the pharmaceutical formulation is suitable for subcutaneous, intravenous, or intravitreal administration. In some embodiments, the pharmaceutical formulation is suitable for subcutaneous or intravitreal administration.

A. Antibody Preparation

The antibody in the liquid formulations provided herein is directed against an antigen of interest. Preferably, the antigen is a biologically important polypeptide and administration of the antibody to a mammal suffering from a disorder can result in a therapeutic benefit in that mammal. However, antibodies directed against non-polypeptide antigens are also contemplated.

Where the antigen is a polypeptide, it may be a transmembrane molecule (e.g. receptor) or ligand such as a growth factor. Exemplary antigens include molecules such as vascular endothelial growth factor (VEGF); CD20; ox-LDL; ox-ApoB100; renin; a growth hormone, including human growth hormone and bovine growth hormone; growth hormone releasing factor; parathyroid hormone; thyroid stimulating hormone; lipoproteins; alpha-1-antitrypsin; insulin A-chain; insulin B-chain; proinsulin; follicle stimulating hormone; calcitonin; luteinizing hormone; glucagon; clotting factors such as factor VIIIC, factor IX, tissue factor, and von Willebrands factor; anti-clotting factors such as Protein C; atrial natriuretic factor; lung surfactant; a plasminogen activator, such as urokinase or human urine or tissue-type plasminogen activator (t-PA); bombesin; thrombin; hematopoietic growth factor; a tumor necrosis factor receptor such as death receptor 5 and CD120; tumor necrosis factor-alpha and -beta; enkephalinase; RANTES (regulated on activation normally T-cell expressed and secreted); human macrophage inflammatory protein (MIP-1-alpha); a serum albumin such as human serum albumin; Muellerian-inhibiting substance; relaxin A-chain; relaxin B-chain; prorelaxin; mouse gonadotropin-associated peptide; a microbial protein, such as beta-lactamase; DNase; IgE; a cytotoxic T-lymphocyte associated antigen (CTLA), such as CTLA-4; inhibin; activin; receptors for hormones or growth factors; protein A or D; rheumatoid factors; a neurotrophic factor such as bone-derived neurotrophic factor (BDNF), neurotrophin-3, -4, -5, or -6 (NT-3, NT4, NT-5, or NT-6), or a nerve growth factor such as NGF-β; platelet-derived growth factor (PDGF); fibroblast growth factor such as aFGF and bFGF; epidermal growth factor (EGF); transforming growth factor (TGF) such as TGF-alpha and TGF-beta, including TGF-β1, TGF-β2, TGF-β3, TGF-β4, or TGF-β5; insulin-like growth factor-I and -II (IGF-I and IGF-II); des (1-3)-IGF-I (brain IGF-I), insulin-like growth factor binding proteins; CD proteins such as CD3, CD4, CD8, CD19 and CD20; erythropoietin; osteoinductive factors; immunotoxins; a bone morphogenetic protein (BMP); an interferon such as interferon-alpha, -beta, and -gamma; colony stimulating factors (CSFs), e.g., M-CSF, GM-CSF, and G-CSF; interleukins (ILs), e.g., IL-1 to IL-10; superoxide dismutase; T-cell receptors; surface membrane proteins; decay accelerating factor; viral antigen such as, for example, a portion of the AIDS envelope; transport proteins; homing receptors; addressins; regulatory proteins; integrins such as CD11a, CD11b, CD11c, CD18, an ICAM, VLA-4 and VCAM; a tumor associated antigen such as HER2, HER3 or HERO receptor; and fragments of any of the above-listed polypeptides.

(i) Antigen Preparation

Soluble antigens or fragments thereof, optionally conjugated to other molecules, can be used as immunogens for generating antibodies. For transmembrane molecules, such as receptors, fragments of these (e.g. the extracellular domain of a receptor) can be used as the immunogen. Alternatively, cells expressing the transmembrane molecule can be used as the immunogen. Such cells can be derived from a natural source (e.g. cancer cell lines) or may be cells which have been transformed by recombinant techniques to express the transmembrane molecule. Other antigens and forms thereof useful for preparing antibodies will be apparent to those in the art.

(ii) Certain Antibody-Based Methods

Polyclonal antibodies are preferably raised in animals by multiple subcutaneous (sc) or intraperitoneal (ip) injections of the relevant antigen and an adjuvant. It may be useful to conjugate the relevant antigen to a protein that is immunogenic in the species to be immunized, e.g., keyhole limpet hemocyanin, serum albumin, bovine thyroglobulin, or soybean trypsin inhibitor using a bifunctional or derivatizing agent, for example, maleimidobenzoyl sulfosuccinimide ester (conjugation through cysteine residues), N-hydroxysuccinimide (through lysine residues), glutaraldehyde, succinic anhydride, SOCl₂, or R¹N═C═NR, where R and R′ are different alkyl groups.

Animals are immunized against the antigen, immunogenic conjugates, or derivatives by combining, e.g., 100 μg or 5 μg of the protein or conjugate (for rabbits or mice, respectively) with 3 volumes of Freund's complete adjuvant and injecting the solution intradermally at multiple sites. One month later the animals are boosted with ⅕ to 1/10 the original amount of peptide or conjugate in Freund's complete adjuvant by subcutaneous injection at multiple sites. Seven to 14 days later the animals are bled and the serum is assayed for antibody titer. Animals are boosted until the titer plateaus. Preferably, the animal is boosted with the conjugate of the same antigen, but conjugated to a different protein and/or through a different cross-linking reagent. Conjugates also can be made in recombinant cell culture as protein fusions. Also, aggregating agents such as alum are suitably used to enhance the immune response.

Monoclonal antibodies of interest can be made using the hybridoma method first described by Kohler et al., Nature, 256:495 (1975), and further described, e.g., in Hongo et al., Hybridoma, 14 (3): 253-260 (1995), Harlow et al., Antibodies: A Laboratory Manual, (Cold Spring Harbor Laboratory Press, 2nd ed. 1988); Hammerling et al., in: Monoclonal Antibodies and T-Cell Hybridomas 563-681 (Elsevier, N.Y., 1981), and Ni, Xiandai Mianyixue, 26(4):265-268 (2006) regarding human-human hybridomas. Additional methods include those described, for example, in U.S. Pat. No. 7,189,826 regarding production of monoclonal human natural IgM antibodies from hybridoma cell lines. Human hybridoma technology (Trioma technology) is described in Vollmers and Brandlein, Histology and Histopathology, 20(3):927-937 (2005) and Vollmers and Brandlein, Methods and Findings in Experimental and Clinical Pharmacology, 27(3):185-91 (2005).

For various other hybridoma techniques, see, e.g., US 2006/258841; US 2006/183887 (fully human antibodies), US 2006/059575; US 2005/287149; US 2005/100546; US 2005/026229; and U.S. Pat. Nos. 7,078,492 and 7,153,507. An exemplary protocol for producing monoclonal antibodies using the hybridoma method is described as follows. In one embodiment, a mouse or other appropriate host animal, such as a hamster, is immunized to elicit lymphocytes that produce or are capable of producing antibodies that will specifically bind to the protein used for immunization. Antibodies are raised in animals by multiple subcutaneous (sc) or intraperitoneal (ip) injections of a polypeptide of interest or a fragment thereof, and an adjuvant, such as monophosphoryl lipid A (MPL)/trehalose dicrynomycolate (TDM) (Ribi Immunochem. Research, Inc., Hamilton, Mont.). A polypeptide of interest (e.g., antigen) or a fragment thereof may be prepared using methods well known in the art, such as recombinant methods, some of which are further described herein. Serum from immunized animals is assayed for anti-antigen antibodies, and booster immunizations are optionally administered. Lymphocytes from animals producing anti-antigen antibodies are isolated. Alternatively, lymphocytes may be immunized in vitro.

Lymphocytes are then fused with myeloma cells using a suitable fusing agent, such as polyethylene glycol, to form a hybridoma cell. See, e.g., Goding, Monoclonal Antibodies: Principles and Practice, pp. 59-103 (Academic Press, 1986). Myeloma cells may be used that fuse efficiently, support stable high-level production of antibody by the selected antibody-producing cells, and are sensitive to a medium such as HAT medium. Exemplary myeloma cells include, but are not limited to, murine myeloma lines, such as those derived from MOPC-21 and MPC-11 mouse tumors available from the Salk Institute Cell Distribution Center, San Diego, Calif. USA, and SP-2 or X63-Ag8-653 cells available from the American Type Culture Collection, Rockville, Md. USA. Human myeloma and mouse-human heteromyeloma cell lines also have been described for the production of human monoclonal antibodies (Kozbor, J. Immunol., 133:3001 (1984); Brodeur et al., Monoclonal Antibody Production Techniques and Applications, pp. 51-63 (Marcel Dekker, Inc., New York, 1987)).

The hybridoma cells thus prepared are seeded and grown in a suitable culture medium, e.g., a medium that contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells. Preferably, serum-free hybridoma cell culture methods are used to reduce use of animal-derived serum such as fetal bovine serum, as described, for example, in Even et al., Trends in Biotechnology, 24(3), 105-108 (2006).

Oligopeptides as tools for improving productivity of hybridoma cell cultures are described in Franek, Trends in Monoclonal Antibody Research, 111-122 (2005). Specifically, standard culture media are enriched with certain amino acids (alanine, serine, asparagine, proline), or with protein hydrolysate fractions, and apoptosis may be significantly suppressed by synthetic oligopeptides, constituted of three to six amino acid residues. The peptides are present at millimolar or higher concentrations.

Culture medium in which hybridoma cells are growing may be assayed for production of monoclonal antibodies that bind to an antibody described herein. The binding specificity of monoclonal antibodies produced by hybridoma cells may be determined by immunoprecipitation or by an in vitro binding assay, such as radioimmunoassay (RIA) or enzyme-linked immunoadsorbent assay (ELISA). The binding affinity of the monoclonal antibody can be determined, for example, by Scatchard analysis. See, e.g., Munson et al., Anal. Biochem., 107:220 (1980).

After hybridoma cells are identified that produce antibodies of the desired specificity, affinity, and/or activity, the clones may be subcloned by limiting dilution procedures and grown by standard methods. See, e.g., Goding, supra. Suitable culture media for this purpose include, for example, D-MEM or RPMI-1640 medium. In addition, hybridoma cells may be grown in vivo as ascites tumors in an animal. Monoclonal antibodies secreted by the subclones are suitably separated from the culture medium, ascites fluid, or serum by conventional immunoglobulin purification procedures such as, for example, protein A-Sepharose, hydroxylapatite chromatography, gel electrophoresis, dialysis, or affinity chromatography. One procedure for isolation of proteins from hybridoma cells is described in US 2005/176122 and U.S. Pat. No. 6,919,436. The method includes using minimal salts, such as lyotropic salts, in the binding process and preferably also using small amounts of organic solvents in the elution process.

(iii) Certain Library Screening Methods

Antibodies in the formulations and compositions described herein can be made by using combinatorial libraries to screen for antibodies with the desired activity or activities. For example, a variety of methods are known in the art for generating phage display libraries and screening such libraries for antibodies possessing the desired binding characteristics. Such methods are described generally in Hoogenboom et al. in Methods in Molecular Biology 178:1-37 (O'Brien et al., ed., Human Press, Totowa, N.J., 2001). For example, one method of generating antibodies of interest is through the use of a phage antibody library as described in Lee et al., J. Mol. Biol. (2004), 340(5):1073-93.

In principle, synthetic antibody clones are selected by screening phage libraries containing phage that display various fragments of antibody variable region (Fv) fused to phage coat protein. Such phage libraries are panned by affinity chromatography against the desired antigen. Clones expressing Fv fragments capable of binding to the desired antigen are adsorbed to the antigen and thus separated from the non-binding clones in the library. The binding clones are then eluted from the antigen, and can be further enriched by additional cycles of antigen adsorption/elution. Any of the antibodies can be obtained by designing a suitable antigen screening procedure to select for the phage clone of interest followed by construction of a full length antibody clone using the Fv sequences from the phage clone of interest and suitable constant region (Fc) sequences described in Kabat et al., Sequences of Proteins of Immunological Interest, Fifth Edition, NIH Publication 91-3242, Bethesda Md. (1991), vols. 1-3.

In some embodiments, the antigen-binding domain of an antibody is formed from two variable (V) regions of about 110 amino acids, one each from the light (VL) and heavy (VH) chains, that both present three hypervariable loops (HVRs) or complementarity-determining regions (CDRs). Variable domains can be displayed functionally on phage, either as single-chain Fv (scFv) fragments, in which VH and VL are covalently linked through a short, flexible peptide, or as Fab fragments, in which they are each fused to a constant domain and interact non-covalently, as described in Winter et al., Ann. Rev. Immunol., 12: 433-455 (1994). As used herein, scFv encoding phage clones and Fab encoding phage clones are collectively referred to as “Fv phage clones” or “Fv clones.”

Repertoires of VH and VL genes can be separately cloned by polymerase chain reaction (PCR) and recombined randomly in phage libraries, which can then be searched for antigen-binding clones as described in Winter et al., Ann. Rev. Immunol., 12: 433-455 (1994). Libraries from immunized sources provide high-affinity antibodies to the immunogen without the requirement of constructing hybridomas. Alternatively, the naive repertoire can be cloned to provide a single source of human antibodies to a wide range of non-self and also self-antigens without any immunization as described by Griffiths et al., EMBO J, 12: 725-734 (1993). Finally, naive libraries can also be made synthetically by cloning the unrearranged V-gene segments from stem cells, and using PCR primers containing random sequence to encode the highly variable CDR3 regions and to accomplish rearrangement in vitro as described by Hoogenboom and Winter, J. Mol. Biol., 227: 381-388 (1992).

In some embodiments, filamentous phage is used to display antibody fragments by fusion to the minor coat protein pIII. The antibody fragments can be displayed as single chain Fv fragments, in which VH and VL domains are connected on the same polypeptide chain by a flexible polypeptide spacer, e.g. as described by Marks et al., J. Mol. Biol., 222: 581-597 (1991), or as Fab fragments, in which one chain is fused to pIII and the other is secreted into the bacterial host cell periplasm where assembly of a Fab-coat protein structure which becomes displayed on the phage surface by displacing some of the wild type coat proteins, e.g. as described in Hoogenboom et al., Nucl. Acids Res., 19: 4133-4137 (1991).

In general, nucleic acids encoding antibody gene fragments are obtained from immune cells harvested from humans or animals. If a library biased in favor of anti-antigen clones is desired, the subject is immunized with antigen to generate an antibody response, and spleen cells and/or circulating B cells other peripheral blood lymphocytes (PBLs) are recovered for library construction. In one embodiment, a human antibody gene fragment library biased in favor of anti-antigen clones is obtained by generating an anti-antigen antibody response in transgenic mice carrying a functional human immunoglobulin gene array (and lacking a functional endogenous antibody production system) such that antigen immunization gives rise to B cells producing human antibodies against antigen. The generation of human antibody-producing transgenic mice is described below.

Additional enrichment for anti-antigen reactive cell populations can be obtained by using a suitable screening procedure to isolate B cells expressing antigen-specific membrane bound antibody, e.g., by cell separation using antigen affinity chromatography or adsorption of cells to fluorochrome-labeled antigen followed by flow-activated cell sorting (FACS).

Alternatively, the use of spleen cells and/or B cells or other PBLs from an unimmunized donor provides a better representation of the possible antibody repertoire, and also permits the construction of an antibody library using any animal (human or non-human) species in which antigen is not antigenic. For libraries incorporating in vitro antibody gene construction, stem cells are harvested from the subject to provide nucleic acids encoding unrearranged antibody gene segments. The immune cells of interest can be obtained from a variety of animal species, such as human, mouse, rat, lagomorpha, luprine, canine, feline, porcine, bovine, equine, and avian species, etc.

Nucleic acid encoding antibody variable gene segments (including VH and VL segments) are recovered from the cells of interest and amplified. In the case of rearranged VH and VL gene libraries, the desired DNA can be obtained by isolating genomic DNA or mRNA from lymphocytes followed by polymerase chain reaction (PCR) with primers matching the 5′ and 3′ ends of rearranged VH and VL genes as described in Orlandi et al., Proc. Natl. Acad. Sci. (USA), 86: 3833-3837 (1989), thereby making diverse V gene repertoires for expression. The V genes can be amplified from cDNA and genomic DNA, with back primers at the 5′ end of the exon encoding the mature V-domain and forward primers based within the J-segment as described in Orlandi et al. (1989) and in Ward et al., Nature, 341: 544-546 (1989). However, for amplifying from cDNA, back primers can also be based in the leader exon as described in Jones et al., Biotechnol., 9: 88-89 (1991), and forward primers within the constant region as described in Sastry et al., Proc. Natl. Acad. Sci. (USA), 86: 5728-5732 (1989). To maximize complementarity, degeneracy can be incorporated in the primers as described in Orlandi et al. (1989) or Sastry et al. (1989). In some embodiments, library diversity is maximized by using PCR primers targeted to each V-gene family in order to amplify all available VH and VL arrangements present in the immune cell nucleic acid sample, e.g. as described in the method of Marks et al., J. Mol. Biol. 222: 581-597 (1991) or as described in the method of Orum et al., Nucleic Acids Res., 21: 4491-4498 (1993). For cloning of the amplified DNA into expression vectors, rare restriction sites can be introduced within the PCR primer as a tag at one end as described in Orlandi et al. (1989), or by further PCR amplification with a tagged primer as described in Clackson et al., Nature, 352: 624-628 (1991).

Repertoires of synthetically rearranged V genes can be derived in vitro from V gene segments. Most of the human VH-gene segments have been cloned and sequenced (reported in Tomlinson et al., J. Mol. Biol., 227: 776-798 (1992)), and mapped (reported in Matsuda et al., Nature Genet., 3: 88-94 (1993); these cloned segments (including all the major conformations of the H1 and H2 loop) can be used to generate diverse VH gene repertoires with PCR primers encoding H3 loops of diverse sequence and length as described in Hoogenboom and Winter, J. Mol. Biol., 227: 381-388 (1992). VH repertoires can also be made with all the sequence diversity focused in a long H3 loop of a single length as described in Barbas et al., Proc. Natl. Acad. Sci. USA, 89: 4457-4461 (1992). Human Vκ and Vλ segments have been cloned and sequenced (reported in Williams and Winter, Eur. J. Immunol., 23: 1456-1461 (1993)) and can be used to make synthetic light chain repertoires. Synthetic V gene repertoires, based on a range of VH and VL folds, and L3 and H3 lengths, will encode antibodies of considerable structural diversity. Following amplification of V-gene encoding DNAs, germline V-gene segments can be rearranged in vitro according to the methods of Hoogenboom and Winter, J. Mol. Biol., 227: 381-388 (1992).

Repertoires of antibody fragments can be constructed by combining VH and VL gene repertoires together in several ways. Each repertoire can be created in different vectors, and the vectors recombined in vitro, e.g., as described in Hogrefe et al., Gene, 128: 119-126 (1993), or in vivo by combinatorial infection, e.g., the loxP system described in Waterhouse et al., Nucl. Acids Res., 21: 2265-2266 (1993). The in vivo recombination approach exploits the two-chain nature of Fab fragments to overcome the limit on library size imposed by E. coli transformation efficiency. Naive VH and VL repertoires are cloned separately, one into a phagemid and the other into a phage vector. The two libraries are then combined by phage infection of phagemid-containing bacteria so that each cell contains a different combination and the library size is limited only by the number of cells present (about 10¹² clones). Both vectors contain in vivo recombination signals so that the VH and VL genes are recombined onto a single replicon and are co-packaged into phage virions. These huge libraries provide large numbers of diverse antibodies of good affinity (K_{d}⁻¹ of about 10⁻⁸M).

Alternatively, the repertoires may be cloned sequentially into the same vector, e.g. as described in Barbas et al., Proc. Natl. Acari Sci. USA, 88: 7978-7982 (1991), or assembled together by PCR and then cloned, e.g. as described in Clackson et al., Nature, 352: 624-628 (1991). PCR assembly can also be used to join VH and VL DNAs with DNA encoding a flexible peptide spacer to form single chain Fv (scFv) repertoires. In yet another technique, “in cell PCR assembly” is used to combine VH and VL genes within lymphocytes by PCR and then clone repertoires of linked genes as described in Embleton et al., Nucl. Acids Res., 20: 3831-3837 (1992).

The antibodies produced by naive libraries (either natural or synthetic) can be of moderate affinity (K_{d}⁻¹ of about 10⁶ to 10⁷ M⁻¹), but affinity maturation can also be mimicked in vitro by constructing and reselecting from secondary libraries as described in Winter et al. (1994), supra. For example, mutation can be introduced at random in vitro by using error-prone poly merase (reported in Leung et al., Technique 1: 11-15 (1989)) in the method of Hawkins et al., J. Mol. Biol., 226: 889-896 (1992) or in the method of Gram et al., Proc. Natl. Acad. Sci USA, 89: 3576-3580 (1992). Additionally, affinity maturation can be performed by randomly mutating one or more CDRs, e.g. using PCR with primers carrying random sequence spanning the CDR of interest, in selected individual Fv clones and screening for higher affinity clones. WO 9607754 (published 14 Mar. 1996) described a method for inducing mutagenesis in a complementarity determining region of an immunoglobulin light chain to create a library of light chain genes. Another effective approach is to recombine the VH or VL domains selected by phage display with repertoires of naturally occurring V domain variants obtained from unimmunized donors and screen for higher affinity in several rounds of chain reshuffling as described in Marks et al., Biotechnol., 10: 779-783 (1992). This technique allows the production of antibodies and antibody fragments with affinities of about 10⁻⁹ M or less.

Screening of the libraries can be accomplished by various techniques known in the art. For example, antigen can be used to coat the wells of adsorption plates, expressed on host cells affixed to adsorption plates or used in cell sorting, or conjugated to biotin for capture with streptavidin-coated beads, or used in any other method for panning phage display libraries.

The phage library samples are contacted with immobilized antigen under conditions suitable for binding at least a portion of the phage particles with the adsorbent. Normally, the conditions, including pH, ionic strength, temperature and the like are selected to mimic physiological conditions. The phages bound to the solid phase are washed and then eluted by acid, e.g. as described in Barbas et al., Proc. Natl. Acad. Sci USA, 88: 7978-7982 (1991), or by alkali, e.g. as described in Marks et al., J. Mol. Biol., 222: 581-597 (1991), or by antigen competition, e.g. in a procedure similar to the antigen competition method of Clackson et al., Nature, 352: 624-628 (1991). Phages can be enriched 20-1,000-fold in a single round of selection. Moreover, the enriched phages can be grown in bacterial culture and subjected to further rounds of selection.

The efficiency of selection depends on many factors, including the kinetics of dissociation during washing, and whether multiple antibody fragments on a single phage can simultaneously engage with antigen. Antibodies with fast dissociation kinetics (and weak binding affinities) can be retained by use of short washes, multivalent phage display and high coating density of antigen in solid phase. The high density not only stabilizes the phage through multivalent interactions, but favors rebinding of phage that has dissociated. The selection of antibodies with slow dissociation kinetics (and good binding affinities) can be promoted by use of long washes and monovalent phage display as described in Bass et al., Proteins, 8: 309-314 (1990) and in WO 92/09690, and a low coating density of antigen as described in Marks et al., Biotechnol., 10: 779-783 (1992).

It is possible to select between phage antibodies of different affinities, even with affinities that differ slightly, for antigen. However, random mutation of a selected antibody (e.g. as performed in some affinity maturation techniques) is likely to give rise to many mutants, most binding to antigen, and a few with higher affinity. With limiting antigen, rare high affinity phage could be competed out. To retain all higher affinity mutants, phages can be incubated with excess biotinylated antigen, but with the biotinylated antigen at a concentration of lower molarity than the target molar affinity constant for antigen. The high affinity-binding phages can then be captured by streptavidin-coated paramagnetic beads. Such “equilibrium capture” allows the antibodies to be selected according to their affinities of binding, with sensitivity that permits isolation of mutant clones with as little as two-fold higher affinity from a great excess of phages with lower affinity. Conditions used in washing phages bound to a solid phase can also be manipulated to discriminate on the basis of dissociation kinetics.

Anti-antigen clones may be selected based on activity. In some embodiments, the present disclosure provides anti-antigen antibodies that bind to living cells that naturally express antigen or bind to free floating antigen or antigen attached to other cellular structures. Fv clones corresponding to such anti-antigen antibodies can be selected by: (1) isolating anti-antigen clones from a phage library as described above, and optionally amplifying the isolated population of phage clones by growing up the population in a suitable bacterial host; (2) selecting antigen and a second protein against which blocking and non-blocking activity, respectively, is desired; (3) adsorbing the anti-antigen phage clones to immobilized antigen; (4) using an excess of the second protein to elute any undesired clones that recognize antigen-binding determinants which overlap or are shared with the binding determinants of the second protein; and (5) eluting the clones which remain adsorbed following step (4). Optionally, clones with the desired blocking/non-blocking properties can be further enriched by repeating the selection procedures described herein one or more times.

DNA encoding hybridoma-derived monoclonal antibodies or phage display Fv clones is readily isolated and sequenced using conventional procedures (e.g. by using oligonucleotide primers designed to specifically amplify the heavy and light chain coding regions of interest from hybridoma or phage DNA template). Once isolated, the DNA can be placed into expression vectors, which are then transfected into host cells such as E. coli cells, simian COS cells, Chinese hamster ovary (CHO) cells, or myeloma cells that do not otherwise produce immunoglobulin protein, to obtain the synthesis of the desired monoclonal antibodies in the recombinant host cells. Review articles on recombinant expression in bacteria of antibody-encoding DNA include Skerra et al., Curr. Opinion in Immunol., 5: 256 (1993) and Pluckthun, Immunol. Revs, 130: 151 (1992).

DNA encoding the Fv clones can be combined with known DNA sequences encoding heavy chain and/or light chain constant regions (e.g. the appropriate DNA sequences can be obtained from Kabat et al., supra) to form clones encoding full or partial length heavy and/or light chains. It will be appreciated that constant regions of any isotype can be used for this purpose, including IgG, IgM, IgA, IgD, and IgE constant regions, and that such constant regions can be obtained from any human or animal species. An Fv clone derived from the variable domain DNA of one animal (such as human) species and then fused to constant region DNA of another animal species to form coding sequence(s) for “hybrid,” full length heavy chain and/or light chain is included in the definition of “chimeric” and “hybrid” antibody as used herein. In some embodiments, an Fv clone derived from human variable DNA is fused to human constant region DNA to form coding sequence(s) for full- or partial-length human heavy and/or light chains.

DNA encoding anti-antigen antibody derived from a hybridoma can also be modified, for example, by substituting the coding sequence for human heavy- and light-chain constant domains in place of homologous murine sequences derived from the hybridoma clone (e.g. as in the method of Morrison et al., Proc. Natl. Acad. Sci. USA, 81: 6851-6855 (1984)). DNA encoding a hybridoma- or Fv clone-derived antibody or fragment can be further modified by covalently joining to the immunoglobulin coding sequence all or part of the coding sequence for a non-immunoglobulin polypeptide. In this manner, “chimeric” or “hybrid” antibodies are prepared that have the binding specificity of the Fv clone or hybridoma clone-derived antibodies.

(iv) Humanized and Human Antibodies

Various methods for humanizing non-human antibodies are known in the art. For example, a humanized antibody has one or more amino acid residues introduced into it from a source which is non-human. These non-human amino acid residues are often referred to as “import” residues, which are typically taken from an “import” variable domain. Humanization can be essentially performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody. Accordingly, such “humanized” antibodies are chimeric antibodies (U.S. Pat. No. 4,816,567) wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

The choice of human variable domains, both light and heavy, to be used in making the humanized antibodies is very important to reduce antigenicity. According to the so-called “best-fit” method, the sequence of the variable domain of a rodent antibody is screened against the entire library of known human variable-domain sequences. The human sequence which is closest to that of the rodent is then accepted as the human framework (FR) for the humanized antibody (Sims et al., J. Immunol., 151:2296 (1993); Chothia et al., J. Mol. Biol., 196:901 (1987)). Another method uses a particular framework derived from the consensus sequence of all human antibodies of a particular subgroup of light or heavy chains. The same framework may be used for several different humanized antibodies (Carter et al., Proc. Natl. Acad Sci. USA, 89:4285 (1992); Presta et al., J. Immunol., 151:2623 (1993)).

It is further important that antibodies be humanized with retention of high affinity for the antigen and other favorable biological properties. To achieve this goal, according to one embodiment of the method, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the recipient and import sequences so that the desired antibody characteristic, such as increased affinity for the target antigen(s), is achieved. In general, the hypervariable region residues are directly and most substantially involved in influencing antigen binding.

Human antibodies in the formulations and compositions described herein can be constructed by combining Fv clone variable domain sequence(s) selected from human-derived phage display libraries with known human constant domain sequence(s) as described above. Alternatively, human monoclonal antibodies can be made by the hybridoma method. Human myeloma and mouse-human heteromyeloma cell lines for the production of human monoclonal antibodies have been described, for example, by Kozbor J. Immunol., 133: 3001 (1984); Brodeur et al., Monoclonal Antibody Production Techniques and Applications, pp. 51-63 (Marcel Dekker, Inc., New York, 1987); and Boerner et al., J. Immunol., 147: 86 (1991).

It is possible to produce transgenic animals (e.g., mice) that are capable, upon immunization, of producing a full repertoire of human antibodies in the absence of endogenous immunoglobulin production. For example, it has been described that the homozygous deletion of the antibody heavy-chain joining region (J_{H}) gene in chimeric and germ-line mutant mice results in complete inhibition of endogenous antibody production. Transfer of the human germ-line immunoglobulin gene array in such germ-line mutant mice will result in the production of human antibodies upon antigen challenge. See, e.g., Jakobovits et al, Proc. Natl. Acad. Sci. USA, 90:2551 (1993); Jakobovits et al., Nature, 362:255-258 (1993); Bruggermann et al., Year in Immuno., 7:33 (1993); and Duchosal et al. Nature 355:258 (1992).

Gene shuffling can also be used to derive human antibodies from non-human, e.g. rodent, antibodies, where the human antibody has similar affinities and specificities to the starting non-human antibody. According to this method, which is also called “epitope imprinting”, either the heavy or light chain variable region of a non-human antibody fragment obtained by phage display techniques as described herein is replaced with a repertoire of human V domain genes, creating a population of non-human chain/human chain scFv or Fab chimeras. Selection with antigen results in isolation of a non-human chain/human chain chimeric scFv or Fab wherein the human chain restores the antigen binding site destroyed upon removal of the corresponding non-human chain in the primary phage display clone, i.e. the epitope governs (imprints) the choice of the human chain partner. When the process is repeated in order to replace the remaining non-human chain, a human antibody is obtained (see PCT WO 93/06213 published Apr. 1, 1993). Unlike traditional humanization of non-human antibodies by CDR grafting, this technique provides completely human antibodies, which have no FR or CDR residues of non-human origin.

(v) Antibody Fragments

Antibody fragments may be generated by traditional means, such as enzymatic digestion, or by recombinant techniques. In certain circumstances there are advantages of using antibody fragments, rather than whole antibodies. The smaller size of the fragments allows for rapid clearance; and may lead to improved access to solid tumors. For a review of certain antibody fragments, see Hudson et al. (2003) Nat. Med. 9:129-134.

Various techniques have been developed for the production of antibody fragments. Traditionally, these fragments were derived via proteolytic digestion of intact antibodies (see, e.g., Morimoto et al., Journal of Biochemical and Biophysical Methods 24:107-117 (1992); and Brennan et al., Science, 229:81 (1985)). However, these fragments can now be produced directly by recombinant host cells. Fab, Fv and ScFv antibody fragments can all be expressed in and secreted from E. coli, thus allowing the facile production of large amounts of these fragments. Antibody fragments can be isolated from the antibody phage libraries discussed above. Alternatively, Fab′-SH fragments can be directly recovered from E. coli and chemically coupled to form F(ab′)₂ fragments (Carter et al., Bio/Technology 10:163-167 (1992)). According to another approach, F(ab′)₂ fragments can be isolated directly from recombinant host cell culture. Fab and F(ab′) 2 fragment with increased in vivo half-life comprising salvage receptor binding epitope residues are described in U.S. Pat. No. 5,869,046. Other techniques for the production of antibody fragments will be apparent to the skilled practitioner. In some embodiments, an antibody is a single chain Fv fragment (scFv). See WO 93/16185; U.S. Pat. Nos. 5,571,894; and 5,587,458. Fv and scFv are the only species with intact combining sites that are devoid of constant regions; thus, they may be suitable for reduced nonspecific binding during in vivo use. scFv fusion proteins may be constructed to yield fusion of an effector protein at either the amino or the carboxy terminus of an scFv. See Antibody Engineering, ed. Borrebaeck, supra. The antibody fragment may also be a “linear antibody”, e.g., as described in U.S. Pat. No. 5,641,870, for example. Such linear antibodies may be monospecific or bispecific.

(vi) Multispecific Antibodies

Multispecific antibodies have binding specificities for at least two different epitopes, where the epitopes are usually from different antigens. While such molecules normally will only bind two different epitopes (i.e. bispecific antibodies, BsAbs), antibodies with additional specificities such as trispecific antibodies are encompassed by this expression when used herein. Bispecific antibodies can be prepared as full length antibodies or antibody fragments (e.g. F(ab′)₂ bispecific antibodies).

Methods for making bispecific antibodies are known in the art. Traditional production of full length bispecific antibodies is based on the co-expression of two immunoglobulin heavy chain-light chain pairs, where the two chains have different specificities (Millstein et al., Nature, 305:537-539 (1983)). Because of the random assortment of immunoglobulin heavy and light chains, these hybridomas (quadromas) produce a potential mixture of 10 different antibody molecules, of which only one has the correct bispecific structure. Purification of the correct molecule, which is usually done by affinity chromatography steps, is rather cumbersome, and the product yields are low. Similar procedures are disclosed in WO 93/08829, and in Traunecker et al., EMBO 1, 10:3655-3659 (1991).

According to a different approach, antibody variable domains with the desired binding specificities (antibody-antigen combining sites) are fused to immunoglobulin constant domain sequences. The fusion preferably is with an immunoglobulin heavy chain constant domain, comprising at least part of the hinge, CH2, and CH3 regions. It is typical to have the first heavy-chain constant region (CH1) containing the site necessary for light chain binding, present in at least one of the fusions. DNAs encoding the immunoglobulin heavy chain fusions and, if desired, the immunoglobulin light chain, are inserted into separate expression vectors, and are co-transfected into a suitable host organism. This provides for great flexibility in adjusting the mutual proportions of the three polypeptide fragments in embodiments when unequal ratios of the three polypeptide chains used in the construction provide the optimum yields. It is, however, possible to insert the coding sequences for two or all three polypeptide chains in one expression vector when the expression of at least two polypeptide chains in equal ratios results in high yields or when the ratios are of no particular significance.

In one embodiment of this approach, the bispecific antibodies are composed of a hybrid immunoglobulin heavy chain with a first binding specificity in one arm, and a hybrid immunoglobulin heavy chain-light chain pair (providing a second binding specificity) in the other arm. It was found that this asymmetric structure facilitates the separation of the desired bispecific compound from unwanted immunoglobulin chain combinations, as the presence of an immunoglobulin light chain in only one half of the bispecific molecule provides for a facile way of separation. This approach is disclosed in WO 94/04690. For further details of generating bispecific antibodies see, for example, Suresh et al., Methods in Enzymology. 121:210 (1986).

According to another approach described in WO96/27011, the interface between a pair of antibody molecules can be engineered to maximize the percentage of heterodimers which are recovered from recombinant cell culture. One interface comprises at least a part of the C_{H}3 domain of an antibody constant domain. In this method, one or more small amino acid side chains from the interface of the first antibody molecule are replaced with larger side chains (e.g. tyrosine or tryptophan). Compensatory “cavities” of identical or similar size to the large side chain(s) are created on the interface of the second antibody molecule by replacing large amino acid side chains with smaller ones (e.g. alanine or threonine). This provides a mechanism for increasing the yield of the heterodimer over other unwanted end-products such as homodimers.

Bispecific antibodies include cross-linked or “heteroconjugate” antibodies. For example, one of the antibodies in the heteroconjugate can be coupled to avidin, the other to biotin. Such antibodies have, for example, been proposed to target immune system cells to unwanted cells (U.S. Pat. No. 4,676,980), and for treatment of HIV infection (WO 91/00360, WO 92/200373, and EP 03089). Heteroconjugate antibodies may be made using any convenient cross-linking methods. Suitable cross-linking agents are well known in the art, and are disclosed in U.S. Pat. No. 4,676,980, along with a number of cross-linking techniques.

Techniques for generating bispecific antibodies from antibody fragments have also been described in the literature. For example, bispecific antibodies can be prepared using chemical linkage. Brennan et al., Science, 229: 81 (1985) describe a procedure wherein intact antibodies are proteolytically cleaved to generate F(ab′)₂ fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Recent progress has facilitated the direct recovery of Fab′-SH fragments from E. coli, which can be chemically coupled to form bispecific antibodies. Shalaby et al., J. Exp. Med., 175: 217-225 (1992) describe the production of a fully humanized bispecific antibody F(ab′)₂ molecule. Each Fab′ fragment was separately secreted from E. coli and subjected to directed chemical coupling in vitro to form the bispecific antibody.

Various techniques for making and isolating bispecific antibody fragments directly from recombinant cell culture have also been described. For example, bispecific antibodies have been produced using leucine zippers. Kostelny et al., J. Immunol., 148(5):1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA, 90:6444-6448 (1993) has provided an alternative mechanism for making bispecific antibody fragments. The fragments comprise a heavy-chain variable domain (Vu) connected to a light-chain variable domain (V_{L}) by a linker which is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific antibody fragments by the use of single-chain Fv (sFv) dimers has also been reported. See Gruber et al, J. Immunol, 152:5368 (1994).

Antibodies with more than two valencies are contemplated. For example, trispecific antibodies can be prepared. Tuft et al. J. Immunol. 147: 60 (1991).

(vii) Single-Domain Antibodies

In some embodiments, an antibody described herein is a single-domain antibody. A single-domain antibody is a single polypeptide chain comprising all or a portion of the heavy chain variable domain or all or a portion of the light chain variable domain of an antibody. In some embodiments, a single-domain antibody is a human single-domain antibody (Domantis, Inc., Waltham, Mass.; see, e.g., U.S. Pat. No. 6,248,516 B1). In one embodiment, a single-domain antibody consists of all or a portion of the heavy chain variable domain of an antibody.

(viii) Antibody Variants

In some embodiments, amino acid sequence modification(s) of the antibodies described herein are contemplated. For example, it may be desirable to improve the binding affinity and/or other biological properties of the antibody. Amino acid sequence variants of the antibody may be prepared by introducing appropriate changes into the nucleotide sequence encoding the antibody, or by peptide synthesis. Such modifications include, for example, deletions from, and/or insertions into and/or substitutions of, residues within the amino acid sequences of the antibody. Any combination of deletion, insertion, and substitution can be made to arrive at the final construct, provided that the final construct possesses the desired characteristics. The amino acid alterations may be introduced in the subject antibody amino acid sequence at the time that sequence is made.

(ix) Antibody Derivatives

The antibodies in the formulations and compositions of the present disclosure can be further modified to contain additional non-proteinaceous moieties that are known in the art and readily available. In some embodiments, the moieties suitable for derivatization of the antibody are water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1,3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propropylene glycol homopolymers, prolypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymer are attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, etc.

(x) Vectors, Host Cells, and Recombinant Methods

Antibodies may also be produced using recombinant methods. For recombinant production of an anti-antigen antibody, nucleic acid encoding the antibody is isolated and inserted into a replicable vector for further cloning (amplification of the DNA) or for expression. DNA encoding the antibody may be readily isolated and sequenced using conventional procedures (e.g., by using oligonucleotide probes that are capable of binding specifically to genes encoding the heavy and light chains of the antibody). Many vectors are available. The vector components generally include, but are not limited to, one or more of the following: a signal sequence, an origin of replication, one or more marker genes, an enhancer element, a promoter, and a transcription termination sequence.

(a) Signal Sequence Component

An antibody in the formulations and compositions described herein may be produced recombinantly not only directly, but also as a fusion polypeptide with a heterologous polypeptide, which is preferably a signal sequence or other polypeptide having a specific cleavage site at the N-terminus of the mature protein or polypeptide. The heterologous signal sequence selected preferably is one that is recognized and processed (e.g., cleaved by a signal peptidase) by the host cell. For prokaryotic host cells that do not recognize and process a native antibody signal sequence, the signal sequence is substituted by a prokaryotic signal sequence selected, for example, from the group of the alkaline phosphatase, penicillinase, 1pp, or heat-stable enterotoxin II leaders. For yeast secretion the native signal sequence may be substituted by, e.g., the yeast invertase leader, a factor leader (including Saccharomyces and Kluyveromyces α-factor leaders), or acid phosphatase leader, the C. albicans glucoamylase leader, or the signal described in WO 90/13646. In mammalian cell expression, mammalian signal sequences as well as viral secretory leaders, for example, the herpes simplex gD signal, are available.

(b) Origin of Replication

Both expression and cloning vectors contain a nucleic acid sequence that enables the vector to replicate in one or more selected host cells. Generally, in cloning vectors this sequence is one that enables the vector to replicate independently of the host chromosomal DNA, and includes origins of replication or autonomously replicating sequences. Such sequences are well known for a variety of bacteria, yeast, and viruses. The origin of replication from the plasmid pBR322 is suitable for most Gram-negative bacteria, the origin of replication from the 2μ plasmid is suitable for yeast, and various viral origins of replication (SV40, polyoma, adenovirus, VSV or BPV) are useful for cloning vectors in mammalian cells. Generally, the origin of replication component is not needed for mammalian expression vectors (the SV40 origin may typically be used only because it contains the early promoter).

(c) Selection Gene Component

Expression and cloning vectors may contain a selection gene, also termed a selectable marker. Typical selection genes encode proteins that (a) confer resistance to antibiotics or other toxins, e.g., ampicillin, neomycin, methotrexate, or tetracycline, (b) complement auxotrophic deficiencies, or (c) supply critical nutrients not available from complex media, e.g., the gene encoding D-alanine racemase for Bacilli.

One example of a selection scheme utilizes a drug to arrest growth of a host cell. Those cells that are successfully transformed with a heterologous gene produce a protein conferring drug resistance and thus survive the selection regimen. Examples of such dominant selection use the drugs neomycin, mycophenolic acid and hygromycin.

Another example of suitable selectable markers for mammalian cells are those that enable the identification of cells competent to take up antibody-encoding nucleic acid, such as DHFR, glutamine synthetase (GS), thymidine kinase, metallothionein-I and preferably primate metallothionein genes, adenosine deaminase, ornithine decarboxylase, etc.

For example, cells transformed with the DHFR gene are identified by culturing the transformants in a culture medium containing methotrexate (Mtx), a competitive antagonist of DHFR. Under these conditions, the DHFR gene is amplified along with any other co-transformed nucleic acid. A Chinese hamster ovary (CHO) cell line deficient in endogenous DHFR activity (e.g., ATCC CRL-9096) may be used.

Alternatively, cells transformed with the GS gene are identified by culturing the transformants in a culture medium containing L-methionine sulfoximine (Msx), an inhibitor of GS. Under these conditions, the GS gene is amplified along with any other co-transformed nucleic acid. The GS selection/amplification system may be used in combination with the DHFR selection/amplification system described above.

Alternatively, host cells (particularly wild-type hosts that contain endogenous DHFR) transformed or co-transformed with DNA sequences encoding an antibody of interest, wild-type DHFR gene, and another selectable marker such as aminoglycoside 3′-phosphotransferase (APH) can be selected by cell growth in medium containing a selection agent for the selectable marker such as an aminoglycosidic antibiotic, e.g., kanamycin, neomycin, or G418. See U.S. Pat. No. 4,965,199.

A suitable selection gene for use in yeast is the trp1 gene present in the yeast plasmid YRp7 (Stinchcomb et al., Nature, 282:39 (1979)). The trp1 gene provides a selection marker for a mutant strain of yeast lacking the ability to grow in tryptophan, for example, ATCC No. 44076 or PEP4-1. Jones, Genetics, 85:12 (1977). The presence of the trp1 lesion in the yeast host cell genome then provides an effective environment for detecting transformation by growth in the absence of tryptophan. Similarly, Leu2-deficient yeast strains (ATCC 20,622 or 38,626) are complemented by known plasmids bearing the Leu2 gene.

In addition, vectors derived from the 1.6 μm circular plasmid pKD1 can be used for transformation of Kluyveromyces yeasts. Alternatively, an expression system for large-scale production of recombinant calf chymosin was reported for K. lactic. Van den Berg, Bio/Technology, 8:135 (1990). Stable multi-copy expression vectors for secretion of mature recombinant human serum albumin by industrial strains of Kluyveromyces have also been disclosed. Fleer et al., Bio/Technology, 9:968-975 (1991).

(d) Promoter Component

Expression and cloning vectors generally contain a promoter that is recognized by the host organism and is operably linked to nucleic acid encoding an antibody. Promoters suitable for use with prokaryotic hosts include the phoA promoter, β-lactamase and lactose promoter systems, alkaline phosphatase promoter, a tryptophan (trp) promoter system, and hybrid promoters such as the tac promoter. However, other known bacterial promoters are suitable. Promoters for use in bacterial systems also will contain a Shine-Dalgarno (S.D.) sequence operably linked to the DNA encoding an antibody.

Promoter sequences are known for eukaryotes. Virtually all eukaryotic genes have an AT-rich region located approximately 25 to 30 bases upstream from the site where transcription is initiated. Another sequence found 70 to 80 bases upstream from the start of transcription of many genes is a CNCAAT region where N may be any nucleotide. At the 3′ end of most eukaryotic genes is an AATAAA sequence that may be the signal for addition of the poly A tail to the 3′ end of the coding sequence. All of these sequences are suitably inserted into eukaryotic expression vectors.

Examples of suitable promoter sequences for use with yeast hosts include the promoters for 3-phosphoglycerate kinase or other glycolytic enzymes, such as enolase, glyceraldehyde-3-phosphate dehydrogenase, hexokinase, pyruvate decarboxylase, phosphofructokinase, glucose-6-phosphate isomerase, 3-phosphoglycerate mutase, pyruvate kinase, triosephosphate isomerase, phosphoglucose isomerase, and glucokinase.

Other yeast promoters, which are inducible promoters having the additional advantage of transcription controlled by growth conditions, are the promoter regions for alcohol dehydrogenase 2, isocytochrome C, acid phosphatase, degradative enzymes associated with nitrogen metabolism, metallothionein, glyceraldehyde-3-phosphate dehydrogenase, and enzymes responsible for maltose and galactose utilization. Suitable vectors and promoters for use in yeast expression are further described in EP 73,657. Yeast enhancers also are advantageously used with yeast promoters.

Antibody transcription from vectors in mammalian host cells can be controlled, for example, by promoters obtained from the genomes of viruses such as polyoma virus, fowlpox virus, adenovirus (such as Adenovirus 2), bovine papilloma virus, avian sarcoma virus, cytomegalovirus, a retrovirus, hepatitis-B virus, Simian Virus 40 (SV40), or from heterologous mammalian promoters, e.g., the actin promoter or an immunoglobulin promoter, from heat-shock promoters, provided such promoters are compatible with the host cell systems.

The early and late promoters of the SV40 virus are conveniently obtained as an SV40 restriction fragment that also contains the SV40 viral origin of replication. The immediate early promoter of the human cytomegalovirus is conveniently obtained as a HindIII E restriction fragment. A system for expressing DNA in mammalian hosts using the bovine papilloma virus as a vector is disclosed in U.S. Pat. No. 4,419,446. A modification of this system is described in U.S. Pat. No. 4,601,978. See also Reyes et al., Nature 297:598-601 (1982) on expression of human β-interferon cDNA in mouse cells under the control of a thymidine kinase promoter from herpes simplex virus. Alternatively, the Rous Sarcoma Virus long terminal repeat can be used as the promoter.

(e) Enhancer Element Component

Transcription of a DNA encoding an antibody by higher eukaryotes is often increased by inserting an enhancer sequence into the vector. Many enhancer sequences are now known from mammalian genes (globin, elastase, albumin, α-fetoprotein, and insulin). Typically, however, one will use an enhancer from a eukaryotic cell virus. Examples include the SV40 enhancer on the late side of the replication origin (bp 100-270), the cytomegalovirus early promoter enhancer, the polyoma enhancer on the late side of the replication origin, and adenovirus enhancers. See also Yaniv, Nature 297:17-18 (1982) on enhancing elements for activation of eukaryotic promoters. The enhancer may be spliced into the vector at a position 5′ or 3′ to the antibody-encoding sequence, but is preferably located at a site 5′ from the promoter.

(f) Transcription Termination Component

Expression vectors used in eukaryotic host cells (yeast, fungi, insect, plant, animal, human, or nucleated cells from other multicellular organisms) will also contain sequences necessary for the termination of transcription and for stabilizing the mRNA. Such sequences are commonly available from the 5′ and, occasionally 3′, untranslated regions of eukaryotic or viral DNAs or cDNAs. These regions contain nucleotide segments transcribed as polyadenylated fragments in the untranslated portion of the mRNA encoding antibody. One useful transcription termination component is the bovine growth hormone polyadenylation region. See WO94/11026 and the expression vector disclosed therein.

(g) Selection and Transformation of Host Cells

Suitable host cells for cloning or expressing the DNA in the vectors herein are the prokaryote, yeast, or higher eukaryote cells described above. Suitable prokaryotes for this purpose include eubacteria, such as Gram-negative or Gram-positive organisms, for example, Enterobacteriaceae such as Escherichia, e.g., E. coli, Enterobacter, Erwinia, Klebsiella, Proteus, Salmonella, e.g., Salmonella typhimurium, Serratia, e.g, Serratia marcescans, and Shigella, as well as Bacilli such as B. subtilis and B. licheniformis (e.g., B. licheniformis 41P disclosed in DD 266,710 published 12 Apr. 1989), Pseudomonas such as P. aeruginosa, and Streptomyces. One preferred E. coli cloning host is E. coli 294 (ATCC 31,446), although other strains such as E. coli B, E. coli X1776 (ATCC 31,537), and E. coli W3110 (ATCC 27,325) are suitable. These examples are illustrative rather than limiting.

Full length antibody, antibody fusion proteins, and antibody fragments can be produced in bacteria, in particular when glycosylation and Fc effector function are not needed, such as when the therapeutic antibody is conjugated to a cytotoxic agent (e.g., a toxin) that by itself shows effectiveness in tumor cell destruction. Full length antibodies have greater half-life in circulation. Production in E. coli is faster and more cost efficient. For expression of antibody fragments and polypeptides in bacteria, see, e.g., U.S. Pat. No. 5,648,237 (Carter et. al.), U.S. Pat. No. 5,789,199 (Joly et al.), U.S. Pat. No. 5,840,523 (Simmons et al.), which describes translation initiation region (TIR) and signal sequences for optimizing expression and secretion. See also Charlton, Methods in Molecular Biology, Vol. 248 (B. K. C. Lo, ed., Humana Press, Totowa, N.J., 2003), pp. 245-254, describing expression of antibody fragments in E. coli. After expression, the antibody may be isolated from the E. coli cell paste in a soluble fraction and can be purified through, e.g., a protein A or G column depending on the isotype. Final purification can be carried out similar to the process for purifying antibody expressed e.g., in CHO cells.

In addition to prokaryotes, eukaryotic microbes such as filamentous fungi or yeast are suitable cloning or expression hosts for antibody-encoding vectors. Saccharomyces cerevisiae, or common baker's yeast, is the most commonly used among lower eukaryotic host microorganisms. However, a number of other genera, species, and strains are commonly available and useful herein, such as Schizosaccharomyces pombe; Kluyveromyces hosts such as, e.g., K. lactis, K. fragilis (ATCC 12,424), K. bulgaricus (ATCC 16,045), K. wickeramii (ATCC 24,178), K. waltii (ATCC 56,500), K. drosophilarum (ATCC 36,906), K. thermotolerans, and K. marxianus; yarrowia (EP 402,226); Pichia pastoris (EP 183,070); Candida; Trichoderma reesia (EP 244,234); Neurospora crassa; Schwanniomyces such as Schwanniomyces occidentalis; and filamentous fungi such as, e.g., Neurospora, Penicillium, Tolypocladium, and Aspergillus hosts such as A. nidulans and A. niger. For a review discussing the use of yeasts and filamentous fungi for the production of therapeutic proteins, see, e.g., Gemgross, Nat. Biotech. 22:1409-1414 (2004).

Certain fungi and yeast strains may be selected in which glycosylation pathways have been “humanized,” resulting in the production of an antibody with a partially or fully human glycosylation pattern. See, e.g., Li et al., Nat. Biotech. 24:210-215 (2006) (describing humanization of the glycosylation pathway in Pichia pastoris); and Gemgross et al., supra.

Suitable host cells for the expression of glycosylated antibody are also derived from multicellular organisms (invertebrates and vertebrates). Examples of invertebrate cells include plant and insect cells. Numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruitfly), and Bombyx mori have been identified. A variety of viral strains for transfection are publicly available, e.g., the L-1 variant of Autographa californica NPV and the Bm-5 strain of Bombyx mori NPV, and such viruses may be used as the virus herein according to the present disclosure, particularly for transfection of Spodoptera frugiperda cells.

Plant cell cultures of cotton, corn, potato, soybean, petunia, tomato, duckweed (Leninaceae), alfalfa (M. truncatula), and tobacco can also be utilized as hosts. See; e.g., U.S. Pat. Nos. 5,959,177, 6,040,498, 6,420,548, 7,125,978, and 6,417,429 (describing PLANTIBODIES™ technology for producing antibodies in transgenic plants).

Vertebrate cells may be used as hosts, and propagation of vertebrate cells in culture (tissue culture) has become a routine procedure. Examples of useful mammalian host cell lines are monkey kidney CV1 line transformed by SV40 (COS-7, ATCC CRL 1651); human embryonic kidney line (293 or 293 cells subcloned for growth in suspension culture, Graham et al., J. Gen Tirol. 36:59 (1977)); baby hamster kidney cells (BHK, ATCC CCL 10); mouse sertoli cells (TM4, Mather, Biol. Reprod. 23:243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3 Å, ATCC CRL 1442); human lung cells (W138, ATCC CCL 75); human liver cells (Hep G2, HB 8065); mouse mammary tumor (MMT 060562, ATCC CCL51); TRT cells (Mather et al., Annals N.Y. Acad. Sci. 383:44-68 (1982)); MRC 5 cells; FS4 cells; and a human hepatoma line (Hep G2). Other useful mammalian host cell lines include Chinese hamster ovary (CHO) cells, including DHFR⁻ CHO cells (Urlaub et al., Proc. Natl. Acad. Sci. USA 77:4216 (1980)); and myeloma cell lines such as NS0 and Sp2/0. For a review of certain mammalian host cell lines suitable for antibody production, see, e.g., Yazaki and Wu, Methods in Molecular Biology, Vol. 248 (B. K. C. Lo, ed., Humana Press, Totowa, N.J., 2003), pp. 255-268.

Host cells are transformed with the above-described expression or cloning vectors for antibody production and cultured in conventional nutrient media modified as appropriate for inducing promoters, selecting transformants, or amplifying the genes encoding the desired sequences.

(h) Culturing the Host Cells

The host cells used to produce an antibody may be cultured in a variety of media. Commercially available media such as Ham's F10 (Sigma), Minimal Essential Medium ((MEM), (Sigma), RPMI-1640 (Sigma), and Dulbecco's Modified Eagle's Medium ((DMEM), Sigma) are suitable for culturing the host cells. In addition, any of the media described in Ham et al., Meth. Enz. 58:44 (1979), Barnes et al., Anal. Biochem. 102:255 (1980), U.S. Pat. Nos. 4,767,704; 4,657,866; 4,927,762; 4,560,655; or 5,122,469; WO 90/03430; WO 87/00195; or U.S. Pat. Re. 30,985 may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as GENTAMYCIN′ drug), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, and will be apparent to the ordinarily skilled artisan.

(xi) Purification of Antibody

When using recombinant techniques, the antibody can be produced intracellularly, in the periplasmic space, or directly secreted into the medium. If the antibody is produced intracellularly, as a first step, the particulate debris, either host cells or lysed fragments, are removed, for example, by centrifugation or ultrafiltration. Carter et al., Bio/Technology 10:163-167 (1992) describe a procedure for isolating antibodies which are secreted to the periplasmic space of E. coli. Briefly, cell paste is thawed in the presence of sodium acetate (pH 3.5), EDTA, and phenylmethylsulfonylfluoride (PMSF) over about 30 min. Cell debris can be removed by centrifugation. Where the antibody is secreted into the medium, supernatants from such expression systems are generally first concentrated using a commercially available protein concentration filter, for example, an Amicon or Millipore Pellicon ultrafiltration unit. A protease inhibitor such as PMSF may be included in any of the foregoing steps to inhibit proteolysis and antibiotics may be included to prevent the growth of adventitious contaminants.

The antibody composition prepared from the cells can be purified using, for example, hydroxylapatite chromatography, hydrophobic interaction chromatography, gel electrophoresis, dialysis, and affinity chromatography, with affinity chromatography being among one of the typically preferred purification steps. The suitability of protein A as an affinity ligand depends on the species and isotype of any immunoglobulin Fc domain that is present in the antibody. Protein A can be used to purify antibodies that are based on human γ1, γ2, or γ4 heavy chains (Lindmark et al., J. Immunol. Meth. 62:1-13 (1983)). Protein G is recommended for all mouse isotypes and for human γ3 (Guss et al., EMBO J. 5:15671575 (1986)). Protein L can be used to purify antibodies based on the kappa light chain (Nilson et al., J. Immunol. Meth. 164(1):33-40, 1993). The matrix to which the affinity ligand is attached is most often agarose, but other matrices are available. Mechanically stable matrices such as controlled pore glass or poly(styrenedivinyl)benzene allow for faster flow rates and shorter processing times than can be achieved with agarose. Where the antibody comprises a C_{H}3 domain, the Bakerbond ABX™ resin (J. T. Baker, Phillipsburg, N.J.) is useful for purification. Other techniques for protein purification such as fractionation on an ion-exchange column, ethanol precipitation, Reverse Phase HPLC, chromatography on silica, chromatography on heparin SEPHAROSE™ chromatography on an anion or cation exchange resin (such as a polyaspartic acid column); chromatofocusing, SDS-PAGE, and ammonium sulfate precipitation are also available depending on the antibody to be recovered.

In general; various methodologies for preparing antibodies for use in research, testing, and clinical are well-established in the art, consistent with the above-described methodologies and/or as deemed appropriate by one skilled in the art for a particular antibody of interest.

B. Selecting Biologically Active Antibodies

Antibodies produced as described above may be subjected to one or more “biological activity” assays to select an antibody with beneficial properties from a therapeutic perspective. The antibody may be screened for its ability to bind the antigen against which it was raised. For example, for an anti-DR5 antibody (e.g., drozitumab), the antigen binding properties of the antibody can be evaluated in an assay that detects the ability to bind to a death receptor 5 (DR5).

In another embodiment, the affinity of the antibody may be determined by saturation binding; ELISA; and/or competition assays (e.g. RIA's), for example.

Also, the antibody may be subjected to other biological activity assays, e.g., in order to evaluate its effectiveness as a therapeutic. Such assays are known in the art and depend on the target antigen and intended use for the antibody.

To screen for antibodies which bind to a particular epitope on the antigen of interest, a routine cross-blocking assay such as that described in Antibodies, A Laboratory Manual, Cold Spring Harbor Laboratory, Ed Harlow and David Lane (1988), can be performed. Alternatively, epitope mapping, e.g. as described in Champe et al., J. Biol. Chem. 270:1388-1394 (1995), can be performed to determine whether the antibody binds an epitope of interest.

### III. Methods of Preparing the Formulation

Certain aspects of the present disclosure relate to methods of preparing any of the liquid formulations described herein. The liquid formulation may be prepared by mixing the polypeptide having the desired degree of purity with NAT and L-methionine. In some embodiments, the polypeptide to be formulated has not been subjected to prior lyophilization, and the formulation of interest herein is an aqueous formulation. In some embodiments, the polypeptide is a therapeutic protein. In some embodiments, the polypeptide is an antibody. In further embodiments, the antibody is a polyclonal antibody, a monoclonal antibody, a humanized antibody, a human antibody, a chimeric antibody, a multispecific antibody, a bispecific antibody, or an antibody fragment. In some embodiments, the antibody is a full length antibody. In some embodiments, the antibody in the formulation is an antibody fragment, such as an F(ab′)₂, in which case problems that may not occur for the full length antibody (such as clipping of the antibody to Fab) may need to be addressed. The therapeutically effective amount of polypeptide present in the formulation is determined by taking into account the desired dose volumes and mode(s) of administration, for example. Exemplary polypeptide concentrations in the formulation include from about 1 mg/mL to more than about 250 mg/mL, from about 1 mg/mL to about 250 mg/mL, from about 10 mg/mL to about 250 mg/mL, from about 15 mg/mL to about 225 mg/mL, from about 20 mg/mL to about 200 mg/mL, from about 25 mg/mL to about 175 mg/mL, from about 25 mg/mL to about 150 mg/mL, from about 25 mg/mL to about 100 mg/mL, from about 30 mg/mL to about 100 mg/mL or from about 45 mg/mL to about 55 mg/mL. In some embodiments, the polypeptide described herein is susceptible to oxidation. In some embodiments, one or more of the amino acids selected from methionine, cysteine, histidine, tryptophan, and/or tyrosine in the protein is susceptible to oxidation. In some embodiments, one or more tryptophans in the polypeptide are susceptible to oxidation. In some embodiments, one or more methionines in the polypeptide are susceptible to oxidation. In some embodiments, one or more tryptophans and one or more methionines in the polypeptide are susceptible to oxidation.

In some embodiments, the liquid formulation further comprises one or more excipients, such as a stabilizer, a buffer, a surfactant, and/or a tonicity agent. A liquid formulation of the present disclosure is prepared in a pH-buffered solution. The buffer of this present disclosure has a pH in the range from about 4.0 to about 9.0. In some embodiments the pH is in the range from pH 4.0 to 8.5, in the range from pH 4.0 to 8.0, in the range from pH 4.0 to 7.5, in the range from pH 4.0 to 7.0, in the range from pH 4.0 to 6.5, in the range from pH 4.0 to 6.0, in the range from pH 4.0 to 5.5, in the range from pH 4.0 to 5.0, in the range from pH 4.0 to 4.5, in the range from pH 4.5 to 9.0, in the range from pH 5.0 to 9.0, in the range from pH 5.5 to 9.0, in the range from pH 6.0 to 9.0, in the range from pH 6.5 to 9.0, in the range from pH 7.0 to 9.0, in the range from pH 7.5 to 9.0, in the range from pH 8.0 to 9.0, in the range from pH 8.5 to 9.0, in the range from pH 5.7 to 6.8, in the range from pH 5.8 to 6.5, in the range from pH 5.9 to 6.5, in the range from pH 6.0 to 6.5, or in the range from pH 6.2 to 6.5. In some embodiments of the present disclosure, the liquid formulation has a pH of 6.2 or about 6.2. In some embodiments of the present disclosure, the liquid formulation has a pH of 6.0 or about 6.0. In some embodiments of the present disclosure, the liquid formulation has a pH of 5.8 or about 5.8. In some embodiments of the present disclosure, the liquid formulation has a pH of 5.5 or about 5.5. Examples of buffers that will control the pH within this range include organic and inorganic acids and salts thereof. For example, acetate (e.g, histidine acetate, arginine acetate, sodium acetate), succinate histidine succinate, arginine succinate, sodium succinate), gluconate, phosphate, fumarate, oxalate, lactate, citrate, and combinations thereof. The buffer concentration can be from about 1 mM to about 600 mM, depending, for example, on the buffer and the desired isotonicity of the formulation. In some embodiments, the formulation comprises a histidine buffer (e.g., in the concentration from about 5 mM to 100 mM). Examples of histidine buffers include histidine chloride, histidine acetate, histidine phosphate, histidine sulfate, histidine succinate, etc. In some embodiments, histidine in the formulation from about 10 mM to about, 35 mM, about 10 mM to about 30 mM, about 10 mM to about 25 mM, about 10 mM to about 20 mM, about 10 mM to about 15 mM, about 15 mM to about 35 mM, about 20 mM to about 35 mM, about 20 mM to about 30 mM or about 20 mM to about 25 mM. In further embodiments, the arginine in the formulation is from about 50 mM to about 500 mM (e.g., about 100 mM, about 150 mM, or about 200 mM).

The liquid formulation of the present disclosure can further comprise a saccharide, such as a disaccharide (e.g., trehalose or sucrose). A “saccharide” as used herein includes the general composition (CH₂O)n and derivatives thereof, including monosaccharides, disaccharides, trisaccharides, polysaccharides, sugar alcohols, reducing sugars, nonreducing sugars, etc. Examples of saccharides herein include glucose, sucrose, trehalose, lactose, fructose, maltose, dextran, glycerin, dextran, erythritol, glycerol, arabitol, sylitol, sorbitol, mannitol, mellibiose, melezitose, raffinose, mannotriose, stachyose, maltose, lactulose, maltulose, glucitol, maltitol, lactitol, iso-maltulose, etc. In some embodiments, the formulation comprises sucrose.

A surfactant can optionally be added to the liquid formulation. Exemplary surfactants include nonionic surfactants such as polysorbates (e.g. polysorbates 20, 80, etc.) or poloxamers (e.g. poloxamer 188, etc.). The amount of surfactant added is such that it reduces aggregation of the formulated antibody and/or minimizes the formation of particulates in the formulation and/or reduces adsorption. For example, the surfactant may be present in the formulation in an amount from about 0.001% to more than about 1.0%, weight/volume. In some embodiments, the surfactant is present in the formulation in an amount from about 0.001% to about 1.0%, from about 0.001% to about 0.5%, from about 0.005% to about 0.2%, from about 0.01% to about 0.1%, from about 0.02% to about 0.06%, or about 0.03% to about 0.05%, weight/volume. In some embodiments, the surfactant is present in the formulation in an amount of 0.04% or about 0.04%, weight/volume. In some embodiments, the surfactant is present in the formulation in an amount of 0.02% or about 0.02%, weight/volume. In one embodiment, the formulation does not comprise a surfactant.

In one embodiment, the formulation contains the above-identified agents (e.g., antibody, buffer, saccharide, and/or surfactant) and is essentially free of one or more preservatives, such as benzyl alcohol, phenol, m-cresol, chlorobutanol and benzethonium Cl. In another embodiment, a preservative may be included in the formulation, particularly where the formulation is a multidose formulation. The concentration of preservative may be in the range from about 0.1% to about 2%, preferably from about 0.5% to about 1%. One or more other pharmaceutically acceptable carriers, excipients or stabilizers such as those described in Remington's Pharmaceutical Sciences 16th edition, Osol, A. Ed. (1980) may be included in the formulation provided that they do not adversely affect the desired characteristics of the formulation. Exemplary pharmaceutically acceptable excipients herein further include insterstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in US Patent Publication Nos. 2005/0260186 and 2006/0104968. In one aspect, a sHASEGP is combined with one or more additional glycosaminoglycanases such as chondroitinases.

The formulation may further comprise metal ion chelators. Metal ion chelators are well known by those of skill in the art and include, but are not necessarily limited to aminopolycarboxylates, EDTA (ethylenediaminetetraacetic acid), EGTA (ethylene glycol-bis(beta-aminoethyl ether)-N,N,N′,N′-tetraacetic acid), NTA (nitrilotriacetic acid), EDDS (ethylene diamine disuccinate), PDTA (1,3-propylenediaminetetraacetic acid), DTPA (diethylenetriaminepentaacetic acid), ADA (beta-alaninediacetic acid), MGCA (methylglycinediacetic acid), etc. Additionally, some embodiments herein comprise phosphonates/phosphonic acid chelators.

Tonicity agents are present to adjust or maintain the tonicity of liquid in a composition. When used with large, charged biomolecules such as proteins and antibodies, they may also serve as “stabilizers” because they can interact with the charged groups of the amino acid side chains, thereby lessening the potential for inter- and intra-molecular interactions, Tonicity agents can be present in any amount between 0.1% to 25% by weight, or more preferably between 1% to 5% by weight, taking into account the relative amounts of the other ingredients. Preferred tonicity agents include polyhydric sugar alcohols, preferably trihydric or higher sugar alcohols, such as glycerin, erythritol, arabitol, xylitol, sorbitol and mannitol.

The formulations described herein may also contain more than one polypeptide or a small molecule drug as necessary for the particular indication being treated, preferably those with complementary activities that do not adversely affect the other polypeptide. For example, where the antibody is anti-DR5 (e.g., drozitumab), it may be combined with another agent (e.g., a chemotherapeutic agent, and anti-neoplastic agent).

In some embodiments, the formulation is for in vivo administration. In some embodiments, the formulation is sterile. The formulation may be rendered sterile by filtration through sterile filtration membranes. The therapeutic formulations herein generally are placed into a container having a sterile access port, for example, an intravenous solution bag or vial having a stopper pierceable by a hypodermic injection needle. The route of administration is in accordance with known and accepted methods, such as by single or multiple bolus or infusion over a long period of time in a suitable manner, e.g., injection or infusion by subcutaneous, intravenous, intraperitoneal, intramuscular, intraarterial, intralesional, intraarticular, or intravitreal routes, topical administration, inhalation or by sustained release or extended-release means.

The liquid formulation of the present disclosure may be stable upon storage. In some embodiments, the polypeptide in the liquid formulation is stable upon storage at about 0 to about 5° C. (such as about any of 1, 2, 3, or 4° C.) for at least about 12 months (such as at least about any of 15, 18, 21, 24, 27, 30, 33, 36 months, or greater). In some embodiments, the physical stability, chemical stability, or biological activity of the polypeptide in the liquid formulation is evaluated or measured. Any methods known the art may be used to evaluate the stability and biological activity. In some embodiments, the stability is measured by oxidation of the polypeptide in the liquid formulation after storage. Stability can be tested by evaluating physical stability, chemical stability, and/or biological activity of the antibody in the formulation around the time of formulation as well as following storage. Physical and/or stability can be evaluated qualitatively and/or quantitatively in a variety of different ways, including evaluation of aggregate formation (for example using size exclusion chromatography, by measuring turbidity, and/or by visual inspection); by assessing charge heterogeneity using cation exchange chromatography or capillary zone electrophoresis; amino-terminal or carboxy-terminal sequence analysis: mass spectrometric analysis; SDS-PAGE analysis to compare reduced and intact antibody; peptide map (for example tryptic or LYS-C) analysis; evaluating biological activity or antigen binding function of the antibody; etc. Instability may result in aggregation, deamidation (e.g. Asn deamidation), oxidation (e.g. Trp oxidation), isomerization (e.g. Asp isomeriation), clipping/hydrolysis/fragmentation (e.g. hinge region fragmentation), succinimide formation, unpaired cysteine(s), N-terminal extension, C-terminal processing, glycosylation differences, etc. In some embodiments, the oxidation in a protein is determined using one or more of RP-HPLC, LC/MS, or tryptic peptide mapping. In some embodiments, the oxidation in an antibody is determined as a percentage using one or more of RP-HPLC, LC/MS, or tryptic peptide mapping and the formula of:

$$\% Fab Oxidation = 100 \times \frac{Oxidized Fab Peak Area}{Fab Peak Area + Oxidized Fab Peak Area}$$

$$\% Fc Oxidation = 100 \times \frac{Oxidized Fc Peak Area}{Fc Peak Area + Oxidized Fc Peak Area}$$

Also provided herein are methods of making a liquid formulation, or preventing oxidation of a polypeptide in a liquid formulation, comprising adding amounts of NAT and L-methionine that reduce or prevent oxidation of a polypeptide in the liquid formulation. In some embodiments, the liquid formulation comprises an antibody. The amount of the NAT and L-methionine that reduce or prevent oxidation of the polypeptide may be any of the amounts disclosed herein.

### IV. Methods of Reducing Oxidation

Certain aspects of the present disclosure relate to methods of reducing oxidation of a polypeptide (e.g., any of the polypeptides described herein) in a liquid formulation comprising adding an amount of NAT and an amount of L-methionine that reduce or prevent oxidation of the polypeptide in the liquid formulation. In some embodiments, the liquid formulation comprising Nat and L-methionine is any of the liquid formulations described herein. In some embodiments, the polypeptide is susceptible to oxidation. In some embodiments, one or more methionine, cysteine, histidine, tryptophan, and/or tyrosine residues in the polypeptide are susceptible to oxidation. In some embodiments, one or more tryptophan residues in the polypeptide are susceptible to oxidation. In some embodiments, one or more methionine residues in the polypeptide are susceptible to oxidation. In some embodiments, one or more tryptophan and one or more methionine residues in the polypeptide are susceptible to oxidation. In some embodiments, the polypeptide is a therapeutic polypeptide. In some embodiments, the polypeptide is an antibody. In some embodiments, the formulation further comprises at least one additional polypeptide according to any of the polypeptides described herein. In some embodiments, the formulation further comprises one or more excipients. In some embodiments, the formulation is an aqueous formulation. In some embodiments, the formulation is a pharmaceutical formulation (e.g., suitable for administration to a human subject).

For example, a formulation of the present disclosure may comprise a monoclonal antibody, NAT and L-methionine as provided herein which prevent oxidation of the monoclonal antibody (e.g., at one or more tryptophan residues and one or more methionine residues in the antibody), and a buffer that maintains the pH of the formulation to a desirable level. In some embodiments, the formulation has a pH of about 4.5 to about 7.0.

In some embodiments, the amount of NAT added to the formulation is any of the concentrations of NAT provided herein. In some embodiments, the amount of NAT added to the formulation is about 0.3 mM. In some embodiments, the amount of NAT added to the formulation is about 1.0 mM. In some embodiments, the NAT reduces or prevents oxidation of one or more tryptophan residues in the polypeptide (e.g., any of the one or more of the tryptophan residues of an antibody as described herein). In some embodiments, the oxidation of the polypeptide (e.g., the oxidation of one or more tryptophan residues in the polypeptide) is reduced by about 40% to about 100%, such as by about any of 40%, 45%, 50%, 55%, 60%, 65%, 70%, 75%, 80%, 85%, 90%, 95%, 96%, 97%, 98%, 99%, or 100%, including any ranges between these values (e.g., as compared to one or more corresponding tryptophan residues in the polypeptide in a liquid formulation lacking NAT). In some embodiments, no more than about 40% to about 0%, such as no more than about any of 40%, 35%, 30%, 25%, 20%, 15%, 10%, 5%, 4%, 3%, 2%, 1%, or 0%, including any ranges between these values, of the polypeptide is oxidized (e.g., oxidized at one or more tryptophan residues in the polypeptide). In some embodiments, the NAT prevents oxidation of the polypeptide by a reactive oxygen species (ROS).

In some embodiments, the amount of L-methionine added to the formulation is any of the concentrations of L-methionine provided herein. In some embodiments, the amount of L-methionine added to the formulation is about 5.0 mM. In some embodiments, the L-methionine reduces or prevents oxidation of one or more methionine residues in the polypeptide (e.g., any of the one or more of the methionine residues of an antibody as described herein). In some embodiments, the oxidation of the polypeptide (e.g., the oxidation of one or more methionine residues in the polypeptide) is reduced by about 40% to about 100%, such as by about any of 40%, 45%, 50%, 55%, 60%, 65%, 70%, 75%, 80%, 85%, 90%, 95%, 96%, 97%, 98%, 99%, or 100%, including any ranges between these values (e.g., as compared to one or more corresponding methionine residues in the polypeptide in a liquid formulation lacking L-methionine). In some embodiments, no more than about 40% to about 0%, such as no more than about any of 40%, 35%, 30%, 25%, 20%, 15%, 10%, 5%, 4%, 3%, 2%, 1%, or 0%, including any ranges between these values, of the polypeptide is oxidized (e.g., oxidized at one or more methionine residues in the polypeptide). In some embodiments, the L-methionine prevents oxidation of the polypeptide by a reactive oxygen species (ROS).

In some embodiments, the polypeptide (e.g., the antibody) concentration in the formulation is any of the polypeptide concentrations described herein (e.g., about 1 mg/mL to about 250 mg/mL). In some embodiments, the polypeptide is a therapeutic polypeptide. In some embodiments, the polypeptide is an antibody. In some embodiments, the antibody is a polyclonal antibody, a monoclonal antibody, a humanized antibody, a human antibody, a chimeric antibody, a multispecific antibody (e.g., bispecific, trispecific, etc.), or an antibody fragment. In some embodiments, the antibody is derived from an IgG1, IgG2, IgG3, or IgG4 antibody sequence. In some embodiments, the antibody is derived from an IgG1 antibody sequence. In some embodiments, the formulation further comprises one or more excipients. Any suitable excipient known in the art may be used in the formulations described herein, including, for example, a stabilizer, a buffer, a surfactant, a tonicity agent, and any combinations thereof. In some embodiments, the formulation has a pH of about any of the pHs described herein (e.g., about 4.5 to about 7.0).

### V. Administration of the Formulations

Certain aspects of the present disclosure relate to the administration of any of the formulations described herein to a subject. In some embodiments, a liquid formulation of the present disclosure may be used in the preparation of a medicament suitable for administration to a subject (e.g., to treat or prevent cancer in the subject). The liquid formulation may be administered to a subject (e.g., a human) in need of treatment with the polypeptide (e.g., an antibody), in accord with known methods, such as intravenous administration as a bolus or by continuous infusion over a period of time, by intramuscular, intraperitoneal, intracerebrospinal, subcutaneous, intra-articular, intrasynovial, intrathecal, oral, topical, inhalation, or intravitreal routes. In some embodiments, the liquid formulation is administered to the subject by intravenous, intravitreal, or subcutaneous administration. In some embodiments, the liquid formulation is administered to the subject by intravitreal administration. In some embodiments, the liquid formulation is administered to the subject by subcutaneous administration.

The appropriate dosage (“therapeutically effective amount”) of the polypeptide will depend, for example, on the condition to be treated, the severity and course of the condition, whether the polypeptide is administered for preventive or therapeutic purposes, previous therapy, the patient's clinical history and response to the polypeptide, the type of polypeptide used, and the discretion of the attending physician. The polypeptide is suitably administered to the patient at one time or over a series of treatments and may be administered to the patient at any time from diagnosis onwards. The polypeptide may be administered as the sole treatment or in conjunction with other drugs or therapies useful in treating the condition in question. As used herein the term “treatment” refers to both therapeutic treatment and prophylactic or preventative measures. Those in need of treatment include those already with the disorder as well as those in which the disorder is to be prevented. As used herein a “disorder” is any condition that would benefit from treatment including, but not limited to, chronic and acute disorders or diseases including those pathological conditions which predispose the subject to the disorder in question.

In a pharmacological sense, in the context of the present disclosure, a “therapeutically effective amount” of a polypeptide (e.g., an antibody) refers to an amount effective in the prevention or treatment of a disorder for the treatment of which the antibody is effective. In some embodiments, the therapeutically effective amount of the polypeptide administered will be in the range of about 0.1 to about 50 mg/kg (such as about 0.3 to about 20 mg/kg, or about 0.3 to about 15 mg/kg) of patient body weight whether by one or more administrations. In some embodiments, the therapeutically effective amount of the polypeptide is administered as a daily dose, or as multiple daily doses. In some embodiments, the therapeutically effective amount of the polypeptide is administered less frequently than daily, such as weekly or monthly. For example, a polypeptide can be administered at a dose of about 100 to about 400 mg (such as about any of 100, 150, 200, 250, 300, 350, or 400 mg, including any ranges between these values) every one or more weeks (such as every 1, 2, 3, or 4 weeks or more, or every 1, 2, 3, 4, 5, or 6 months or more) or is administered a dose of about 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0, 15.0, or 20.0 mg/kg every one or more weeks (such as every 1, 2, 3, or 4 weeks or more, or every 1, 2, 3, 4, 5, or 6 months or more). The dose may be administered as a single dose or as multiple doses (e.g., 2, 3, 4, or more doses), such as infusions. The progress of this therapy is easily monitored by conventional techniques.

### VI. Articles of Manufacture and Kits

Certain aspects of the present disclosure relate to articles of manufacture or kits comprising a container which holds any of the liquid formulations of the present disclosure. Suitable containers include, for example, bottles, vials and syringes. The container may be formed from a variety of materials such as glass or plastic. An exemplary container is a 2-20 cc single use glass vial. Alternatively, for a multidose formulation, the container may be a 2-100 cc glass vial. The container holds the formulation and the label on, or associated with, the container may indicate directions for use. The article of manufacture may further include other materials desirable from a commercial and user standpoint, including other buffers, diluents, filters, needles, syringes, and package inserts with instructions for use. In some embodiments, the article of manufacture or kit further comprises a package insert comprising instructions for the use of the liquid formulation. A package insert may refer to instructions customarily included in commercial packages of therapeutic products that contain information about the indications, usage, dosage, administration, contraindications and/or warnings concerning the use of such therapeutic products.

Kits are also provided that are useful for various purposes, e.g., for reducing oxidation of a polypeptide in a liquid formulation, or for screening a liquid formulation for reduced oxidation of a polypeptide. Instructions supplied in the kits of the present disclosure are typically written instructions on a label or package insert (e.g., a paper sheet included in the kit), but machine-readable instructions (e.g., instructions carried on a magnetic or optical storage disk) are also acceptable.

The specification is considered to be sufficient to enable one skilled in the art to practice the present disclosure. Various modifications of the present disclosure in addition to those shown and described herein will become apparent to those skilled in the art from the foregoing description and fall within the scope of the appended claims.

### EXAMPLES

The present disclosure will be more fully understood by reference to the following examples. They should not, however, be construed as limiting the scope of the present disclosure. It is understood that the examples and embodiments described herein are for illustrative purposes only and that various modifications or changes in light thereof will be suggested to persons skilled in the art, and are to be included within the spirit and purview of this application and scope of the appended claims.

### Example 1: Assessment of NAT Protection from Oxidation

The following study was conducted to assess the antioxidant efficacy and safety of N-acetyl-DL-tryptophan (NAT) and/or L-methionine as formulation components for biotherapeutics drugs. 2,2′-azo-bis(2-amidinopropane) dihydrochloride (AAPH), an azo compound that generates reactive oxygen species capable of oxidizing both methionine and tryptophan residues (Ji et al. (2009) J. Pharm. Sci. 98(12):4485-4500), as well as light exposure were selected as the oxidation models for the following study, as they represented common oxidation pathways to which antibodies may be exposed during manufacturing and/or long-term storage (Grewal et al. (2014) Mol. Pharm. 11(4):1259-1272).

Materials and Methods

### Materials

MAb1 and mAb2 are IgG1 monoclonal antibodies with oxidation susceptible tryptophan and methionine residues (Dion et al., manuscript in preparation). The mAbs were purified by a series of chromatography steps including Protein A affinity chromatography and ion-exchange chromatography, and formulated in a low ionic strength sodium acetate buffer at pH 5.5 without surfactants or other excipients, unless otherwise specified.

L-Methionine and N-acetyl-DL-tryptophan (NAT) were purchased from Ajinomoto North America (Raleigh, N.C.). 2,2′-azo-bis(2-amidinopropane) dihydrochloride (AAPH) was purchased from Calbiochem (La Jolla, Calif.). Trypsin (mass spectrometry grade) was purchased from Promega (Madison, Wis.). High pressure liquid chromatography (HPLC)-grade acetonitrile and water were purchased from Fisher Scientific (Fairlawn, N.J.). Water used for buffer-preparation was obtained from a Milli-Q purification system (Millipore, Bedford, Mass.).

### Evaluation of NAT Antioxidant Efficacy

### Identification and Monitoring of Oxidation-Sensitive Residues

Antibodies were subjected to AAPH stress followed by peptide mapping to identify the CDR and Fc residues that were sensitive to oxidation (Dion et al., manuscript in preparation). Kabat numbering was used to identify variable fragment (Fv) residues, while EU nomenclature (Edelman et al. (1969) Proc Natl Acad Sci USA 63(1):78-85) was used to identify Fc residues. If a residue oxidized by >5% relative to the control, it was deemed sensitive and monitored throughout the course of the experiments. Peptide mapping and analysis information was as reported in Dion et al. (manuscript in preparation). In brief, samples were denatured, reduced, carboxylmethylated and subjected to trypsin digestion. Peptides were separated on an Acquity UPLC Peptide CSH C18 column using a water/acetonitrile/formic acid gradient on a Waters Acquity H-Class UHPLC coupled to a Thermo Q Exactive Plus high-resolution mass spectrometer. Data was processed using Thermo Scientific PepFinder™ and Xcalibur™ software. Integration was performed on extracted ion chromatograms of monoisotopic m/z using the most abundant charge state(s) for the native and oxidized peptides. The percent oxidation was calculated by dividing the peak area of the oxidized peptides by the summed peak area of the native and oxidized peptides. The major tryptophan degradation products (+16 and +32, in addition to +4, +20, and +48 for highly oxidized sites) were summed and used to calculate tryptophan oxidation. Only methionine sulfoxide (M₊₁₆) was used to calculate methionine oxidation, as methionine sulfone (M₊₃₂) was not observed under these conditions. Where the two software packages provided different answers, Xcalibur™ data was reported after manual checking of the data.

### AAPH Chemical Oxidation Stress Model

Antibodies were prepared to a final concentration of 1 mg/mL in 20 mM sodium acetate, pH 5.5, in 2 cc glass vials. NAT was added to a final concentration of 0.05 mM and 0.3 mM from a stock solution of 3 mM NAT in 20 mM sodium acetate, pH 5.5. L-Methionine was added to a final concentration of 5 mM from a 50 mM stock solution in 20 mM sodium acetate, pH 5.5, for specified samples. AAPH from a stock solution of 11 mM was added to a final concentration of 1 mM. An equivalent volume of water was added to the protein aliquots in place of AAPH for control samples. Following addition of AAPH or water, samples were incubated at 40° C. for 16 h. A control sample was also immediately frozen at −70° C. The free radical-generating reaction was quenched with L-methionine in a ratio of 20:1 L-methionine to AAPH, and each sample was then buffer exchanged into formulation buffer (20 mM sodium acetate, 100 mM sucrose, pH 5.5) using a PD-10 column (GE Healthcare) and concentrated to a final concentration of 10 mg/mL using Amicon Ultra Centrifugal Filters (EMD Millipore) in preparation for analysis via LC-MS peptide mapping.

### Light Exposure Stress Model

Photo-stability studies were conducted by exposing samples at 10 mg/mL in glass vials to light in an Atlas SunTest CPS+Xenon Light box (Chicago, Ill.) with a total dose of 300 kilolux-hours visible light and 50 W·h/m² of near UV (320-400 nm) light. NAT was added to a final concentration of 0.05, 0.1, 0.3, 0.5 or 1.0 mM from the stock solution described previously. Control samples were wrapped in aluminum foil and placed alongside experimental vials. Following exposure, samples were stored at −70° C. in preparation for analysis via LC-MS peptide mapping.

### Safety Assessment of NAT and L-Methionine

### In Silico Mutagenicity and Carcinogenicity Prediction

The mutagenicity and carcinogenicity potential of NAT was assessed using the Derek Nexus (Program version 2.0.2.201111291322; Lhasa Limited, Leeds, UK) and Leadscope® (Model Applier Version 1.5.0; Leadscope Inc., Columbus, Ohio) in silico modeling tools.

### In Vitro Receptor Binding and Function Assessment

The activity of NAT was assessed in binding, cellular and nuclear receptor functional and tissue bioassays. Binding to the neurokinin-1 (NK-1) receptor was assessed in U373MG human astrocytoma cells which endogenously express the receptor (Eistetter et al. (1992) Functional characterization of Neurokinin-1 receptors on human U373MG astrocytoma cells. Glia 6(2):89-95; Heuillet et al. (1993) J. Neurochem 60(3):868-876), and compared to the reference agonist [Sar⁹, Met(O₂)¹¹]-SP or to the reference antagonist L 733,060. NAT or the reference compounds were incubated with U373MG cells at room temperature; all concentrations were assayed in duplicate.

Substance P, acting through the NK-1 receptor, has been shown to modulate vascular tone in both humans and non-clinical species (Coge and Regoli, (1994) Neuropeptides 26(6); 385-390; Shirahase et al. (2000) Br. J. Pharmacol 129(5); 937-942). To assess the potential for specific activity of NAT at the NK-1 receptor, rings of rabbit pulmonary artery with intact endothelium were suspended in 20 mL organ baths filled with an oxygenated (95% O₂/5% CO₂) and pre-warmed (37° C.) physiological salt solution (in mM): NaCl 118.0. KCl 4.7, MgSO₄ 1.2, CaCl₂ 2.5, KH₂PO₄ 1.2, NaHCO₃25 and glucose 11.0 (pH 7.4). Propranolol (1 μM), pyrilamine (1 μM), atropine (1 μM) and methysergide (1 μM) were present throughout the experiments to block the β-adrenergic, histamine H1, muscarinic and 5-HT2 receptors, respectively. The tissues were connected to force transducers for isometric tension recordings, stretched to a resting tension of 2 g, then allowed to equilibrate for 60 minutes during which time they were washed repeatedly and the tension readjusted. The experiments were carried out using semi-automated isolated organ systems possessing eight organ baths, with multichannel data acquisition. The parameter measured was the maximum change in tension induced by each compound concentration.

To evaluate agonist activity, the tissues were contracted with norepinephrine (0.1 μM), exposed to a submaximal concentration of the reference agonist [Sar⁹, Met(O₂)¹¹]-SP (0.001 μM) to verify responsiveness and to obtain a control relaxation, then washed. Thereafter, the tissues were contracted every 45 minutes with norepinephrine, exposed to increasing concentrations of NAT or the reference agonist, then washed. Each compound concentration was left in contact with the tissues until a stable response was obtained or for a maximum of 15 minutes. If an agonist-like response (relaxation) was obtained, the highest concentration of the compound was tested again in the presence of the reference antagonist spantide II (1 μM) added 30 minutes before, to confirm the involvement of the NK1 receptor in this response.

To evaluate antagonist activity, the tissues were contracted with norepinephrine (0.1 μM), exposed to a submaximal concentration of the reference agonist [Sar⁹,Met(O₂)¹¹]-SP (0.001 μM) to obtain a control relaxation, then washed. This sequence was repeated every 45 minutes in the presence of increasing concentrations of NAT or the reference antagonist spantide II, each added 30 minutes before exposure to [Sar⁹,Met(O₂)¹¹]-SP.

### In Vivo Tolerability of NAT/L-Methionine Formulation

All procedures conducted in animals complied with the Animal Welfare Act, the Guide for the Care and Use of Laboratory Animals, and the Office of Laboratory Animal Welfare. Protocols were approved by the applicable Institutional Animal Care and Use Committees.

### Single-Dose Rabbit Intravitreal Tolerability Study

To assess acute tolerability in support of a product intended for the treatment of a retinal disorder, male New Zealand White (NZW) rabbits were administered a single dose of either an isotonic vehicle formulation (n=2) or the vehicle formulation containing 5 mM NAT and 25 mM L-Methionine (n=3) by bilateral intravitreal injection (50 uL/eye).

Animals were dosed with the vehicle solutions on Study Day 1. The assessment of toxicity was based on clinical observations, intraocular pressure (TOP) measurements, and ophthalmic examinations. At necropsy on Day 8, the eyes and optic nerves were collected and processed for hematoxylin and eosin (H&E) stain, and analyzed microscopically by an American College of Veterinary Pathologists (ACVP-certified Veterinary Pathologist.

### Repeat-Dose Rabbit Intravitreal Toxicology Study

A Good Laboratory Practice (GLP) toxicology study in support of a product intended for the treatment of a retinal disorder was conducted in male and female NZW rabbits. Animals (n=5/sex) were administered the vehicle formulation (an isotonic solution containing 1 mM NAT, 5 mM L-methionine at pH 5.5) via bilateral intravitreal injection (50 uL/eye) once every other week (Days 1, 15, 29, and 43). The assessment of toxicity was based on clinical observations, body weight measurements, ophthalmic examinations, IOP measurements, ocular photography, and clinical pathology. At necropsy on Day 45, a comprehensive set of tissues was collected and processed for H&E stain, and analyzed microscopically by an ACVP-certified Veterinary Pathologist.

### Repeat-Dose Cynomolgus Monkey Toxicology Study—Intravitreal Administration

A GLP toxicology study in support of a product intended for the treatment of a retinal disorder was conducted in male and female cynomolgus monkeys (Macaca fascicularis). Animals (n=5/sex) were administered the vehicle formulation (an isotonic solution containing 1 mM NAT, 5 mM L-methionine at pH 5.5) via bilateral intravitreal injection (50 uL/eye) once every other week over a ten week period (Days 1, 15, 29, 43, 57, and 71). The assessment of toxicity was based on clinical observations, physical examinations, electrocardiograms, ophthalmic examinations, spectral domain optical computed tomography (OCT), ocular photography, fluorescein angiography, electroretinography, and clinical pathology. At necropsy on Day 72 or 99, a comprehensive set of tissues was collected and processed for H&E stain, and analyzed microscopically by an ACVP-certified Veterinary Pathologist.

### Repeat-Dose Cynomolgus Monkey Study—Subcutaneous Administration

A GLP toxicology study in support of a product intended for the treatment of metabolic diseases was conducted in male and female cynomolgus monkeys (Macaca fascicularis). Animals (n=8/sex) were administered the vehicle formulation (an isotonic solution containing 0.3 mM NAT, 5 mM L-methionine at pH 5.8) subcutaneously (0.1 mL/kg) once weekly over 4 weeks (Days 1, 8, 15, 22 and 29). The assessment of toxicity was based on clinical observations, physical examinations, neurologic and ophthalmic examinations, clinical pathology, and urinalysis. At necropsy on Day 32 or 99, a comprehensive set of tissues was collected and processed for H&E stain, and analyzed microscopically by an ACVP-certified Veterinary Pathologist.

Results

### AAPH Free Radical Chemical Oxidation Stress

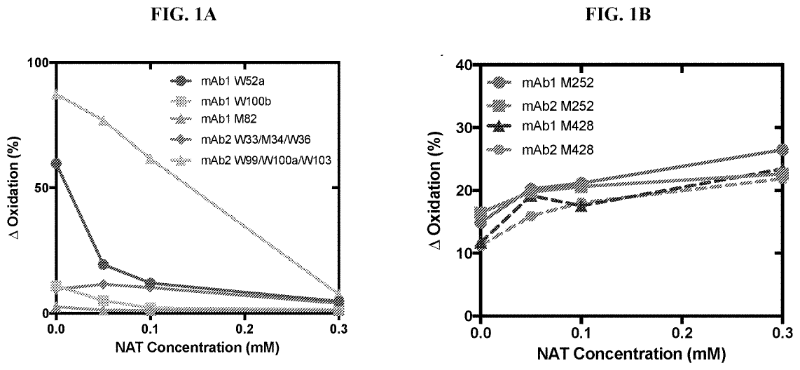

An AAPH stress test was conducted to determine the antioxidant properties of NAT on susceptible tryptophan and methionine residues upon exposure to free radicals in solution. As previously reported (Dion et al., manuscript in preparation), peptide mapping of mAb1 indicated two sensitive CDR tryptophan residues, W52a and W100b, as well as the Fv methionine HC M82. For mAb2, two peptides, each containing multiple sensitive residues, were identified (CDR H1 W33/M34/W36 and CDR H3 W99/W100a and Fv W103). For these two peptides with multiple sensitive residues, the summed oxidation values for each peptide are shown herein. The Fc methionine residues 252 and 428 that interact with the FcRn receptor were also found to be sensitive to oxidative stress in both molecules, consistent with past literature (Bertolotti-Ciarlet et al., 2009). To determine the effect of NAT concentration on antioxidant efficacy, the concentration of NAT in the formulation was varied between 0 mM and 0.3 mM and the formulated mAb subjected to AAPH stress (FIG. 1). With no NAT, the oxidation levels of Fv peptides with sensitive tryptophan residues increased upon AAPH stress by 11% (W100b of mAb1), 60% (W52a of mAb1), and 87% (W99/W100a/W103 of mAb2). These initial starting values gave a broad range of oxidative sensitivity over which to study the impact of NAT. The minimum concentration of NAT required to stabilize tryptophan residues correlated with the initial AAPH sensitivity of the residue (FIG. 1A). Oxidation of mAb1 W100b was reduced to 5% with addition of 0.05 mM NAT, while mAb1 W52a required addition of 0.3 mM NAT to reduce oxidation to 5%. In contrast, oxidation of W99/W100a/W103 of mAb2 was only reduced to 77%, 62% and 8% with addition of 0.05 mM, 0.1 mM and 0.3 mM NAT, respectively. The peptide containing W33/M34/W36 on mAb2 similarly generally decreased with increasing NAT concentration, although the relative effect on the individual tryptophan and methionine residues in that peptide could not be unequivocally determined. The less susceptible M82 residue in mAb 1 was oxidized minimally in the absence of NAT (3%), and inclusion of NAT showed a small effect (slight decrease to 1% oxidation at 0.3 mM NAT).

The impact of NAT concentration on Fc methionine oxidation was also assessed (FIG. 1B). With no NAT, oxidation levels of M252 and M428 for both mAbs were between 11% and 16% after AAPH exposure. In contrast to the CDR residues, which were largely protected from oxidation by NAT, oxidation of Fc methionine residues was exacerbated by the addition of NAT. At the highest level tested (0.3 mM NAT), oxidation of Fc methionine residues increased by 6%-12% relative to the corresponding conditions without NAT.

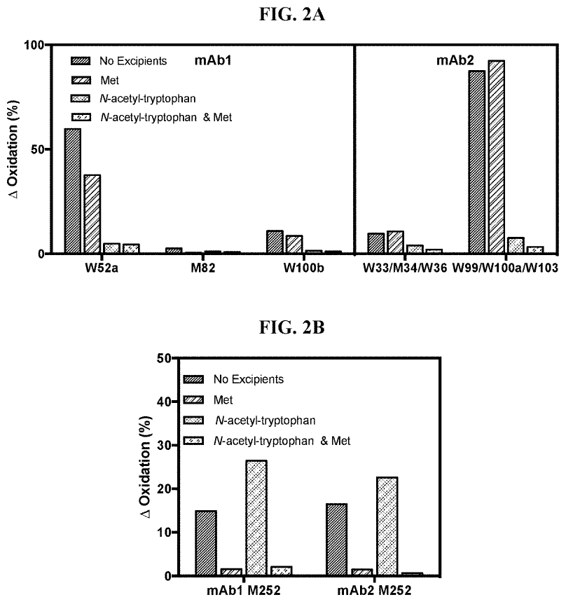

Because NAT protected CDR and Fv tryptophan residues from oxidation (<10% oxidation at 0.3 mM NAT) (FIG. 1A) but exacerbated oxidation of Fc methionine residues (FIG. 1B), an experimental arm including L-methionine co-formulated with NAT was included in the antioxidant efficacy study. L-methionine alone (5 mM) had a mixed effect on AAPH-sensitive tryptophan residues, showing slight improvements in mAb1 and no impact or a slight exacerbation of mAb2 oxidation levels (FIG. 2A). Fc methionine oxidation levels were reduced to 2% or less for both molecules upon addition of L-methionine alone (FIG. 2B). The combination of 0.3 mM NAT and 5 mM L-methionine effectively reduced AAPH-induced oxidation to <5% for CDR tryptophan residues and <2% for Fc methionine residues, making the combination of antioxidant excipients the most effective approach for controlling oxidation levels under the conditions tested (FIGS. 2A-B).

### Light Exposure Stress: High Intensity UV

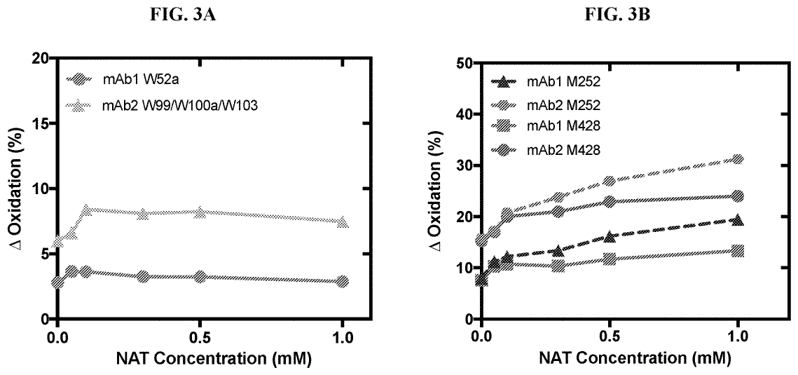

Proteins (10 mg/mL) were exposed to light stress with a high intensity UV component (300 kilolux-hours visible light and 50 W·h/m² of near UV (320-400 nm) light over a 6-hour period) at various NAT concentrations (0-1 mM NAT) to determine the efficacy of NAT as an antioxidant against photo-oxidation (FIGS. 3A-B). A wider NAT concentration range was included, as compared to the AAPH study, based on reports that NAT is photosensitive (Chin et al. (2008) J. Am. Chem. Soc. 130(22):6912-6913). Under the conditions tested, most CDR and Fv residues in mAb1 and mAb2 had oxidation levels ≤1%. Only two peptides showed susceptibility to the tested light conditions (mAb1 W52a (3%) and W99/W100a/W103 of mAb2 (6%)) (FIG. 3A). Oxidation at these sites was minimally impacted by addition of ≥0.1 mM NAT (<1% change for mAb1 W52a, 1-2% increase for W99/W100a/W103 of mAb2). Residues that were determined to be insensitive to light oxidation under antioxidant-free conditions remained insensitive to light when NAT was added to the formulation under the tested conditions.

In contrast to Fv residues, Fc methionine residues were sensitive to UV light stress and to the addition of NAT (FIG. 3B). For example, oxidation of Fc methionine residue M252 increased from 8% without NAT to 19% in the presence of 1 mM NAT in mAb1, and from 16% to 31% for mAb2. These results indicated that, like in the case of AAPH stress, NAT increased the oxidation level of Fc methionine residues under UV light stress conditions.

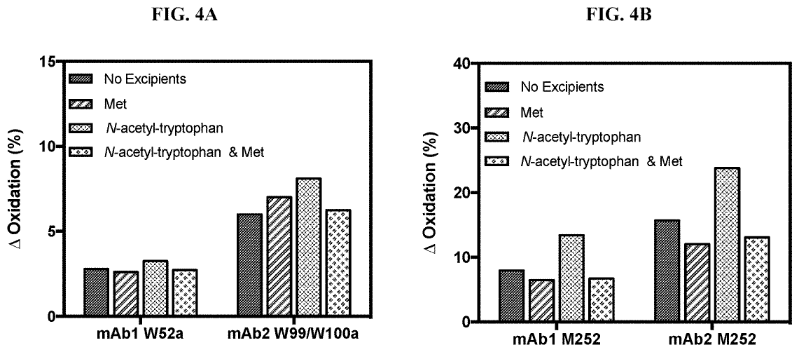

To determine if the sensitization of Fc methionines by NAT could be reduced by the addition of L-methionine, the impact of NAT and L-Methionine individually and in combination under UV light conditions was assessed. The addition of 5 mM L-Methionine, alone or in combination with 0.3 mM NAT, had no beneficial impact on CDR and Fv residues in this oxidation model (FIG. 4A). UV light-induced Fc methionine oxidation was improved by 5 mM L-Methionine (FIG. 4B), but the effect was not as significant as in the AAPH model. The combination of L-Methionine (5 mM) and NAT (0.3 mM) led to minor protection of CDR tryptophans or Fc methionines from photo-oxidation relative to either the no excipient condition or to L-Methionine alone in this strong UV light oxidation model.

### Safety Assessment of NAT and L-Methionine

Given that NAT and methionine are present on the FDA Inactive Ingredient List for parenteral formulations and have been safely used without identification of hazard in acute settings, an abbreviated safety risk assessment was performed to support their use in formulations intended for subcutaneous or intravitreal administration. In vivo tolerability studies of the combination of NAT and L-methionine were performed for both new administration routes. Additionally, as literature reports suggested that NAT might act as an antagonist of the NK-1 receptor, an in silico toxicity assessment and in vitro assessments of NK-1 receptor binding were performed for NAT.

### In Silico Assessment of NAT

Derek is an empirical/rule-based system which derives a prediction by comparing the structural features of the test compound (i.e., NAT) against the portion of molecules in its database thought to be responsible for toxic effects (toxicophores). The structure of NAT was submitted to the Derek Nexus database, which returned a result of “nothing to report”.

Leadscope® is a quantitative structure-activity relationship (QSAR) system which includes pre-trained models for the prediction of genetic toxicity; the system was created in collaboration with the US FDA, and has shown high sensitivity and negative predictivity (Sutter et al. (2013) Reg. Tox. Pharm. 67(1):39-52). Leadscope® assessed the likelihood of a positive result in a total of 40 models. Of these, only 2 models were predicted to be positive, with the remaining 38 predicted to be negative (i.e., no prediction of toxicity). In the Genetic Toxicity category, the “sister chromatid exchange (SCE) in other cells” model was positive with a positive prediction probability of 0.829. In contrast, the two other SCE models (SCE in vitro and SCE in vitro CHO) were both negative. In the Rodent Carcinogenicity category, the “carc mouse male” model was predicted to be positive with a positive prediction probability of 0.622. Prediction probabilities between 0.4-0.6 are considered marginal predictions in the Leadscope® tool. A second run of the model returned a negative prediction, and the overall prediction for mouse carcinogenicity (male and female combined) was negative.

### In Vitro Receptor Binding and Function Assessment

The IC₅₀'s for agonist and antagonist binding of the reference compounds, (Sar⁹, Met(O₂)¹¹)-SP and L 733,060), to the NK-1 receptor were 4.2⁻¹⁰ M and 4.7⁻¹⁰ M, respectively. In contrast, IC₅₀ values could not be calculated for either agonist or antagonist binding of NAT to the NK-1 receptor, indicating a lack of activity of NAT under the conditions employed in the assays.

### In Vivo Tolerability Assessment—Rabbit and Cynomolgus Monkey Toxicology Studies

Vehicle formulations containing up to 5 mM NAT and 25 mM L-methionine were well tolerated by intravitreal administration in rabbits by both single and repeat dose administration for up to 6 weeks. Administration of the vehicle formulation containing 0.3 mM NAT and 1 mM L-methionine was well tolerated in cynomolgus monkeys by intravitreal administration every other week for up to 7 weeks and, similarly, by weekly administration by subcutaneous administration for up to 4 weeks. No vehicle-associated clinical observations or changes in body weight, physical examinations, neurologic or ophthalmic examinations, intraocular pressure, OCT, ocular photography, fluorescein angiography, electroretinography, hematology, coagulation and clinical chemistry parameters, urinalysis, or gross or microscopic pathology were noted in either species.

Taken together, the studies provided herein demonstrated that while NAT was effective at protecting CDR tryptophan residues from ROS produced by AAPH degradation, it may have sensitized Fc methionine residues to chemical and light-induced oxidation. The addition of L-methionine to NAT effectively protected both tryptophan and methionine residues from chemical-induced oxidation, and resulted in photooxidation levels equal to or below those found in formulations without antioxidants for the conditions tested. These studies demonstrated that the combination of NAT and L-methionine was capable of providing protection against the types of oxidation stresses that commonly occur during biotherapeutic manufacturing and storage. Importantly, the safety assessment confirmed that both excipients were well tolerated. Therefore, the evidence presented herein suggested that NAT and L-Methionine may be safe and effective as antioxidant excipients in biotherapeutic formulations, which provides an important new option in formulation development for the management of tryptophan and/or methionine oxidation.

### Example 2. Antioxidants Reduce Oxidation in AAPH Stress Test

Antibody Mab3, a bispecific antibody, was used to evaluate antioxidatation potential of NAT+ methionine. Mab3 was mixed at 1 mg/mL with 1 mM AAPH for 16 hours at 40° C. with or without 1 mM NAT and 5 mM methionine. Oxidation of Mab3 was then measured by mass spectrometry as described above and for potency by ELISA. Results are presented in Table 1.

TABLE 1
Oxidation of Mab3

| Sample | 1 | 2 |
|---|---|---|
| Buffer and pH | His-Ac pH 5.5 | His-Ac pH 5.5 |
| N-acetyl-Trp | — | 1 mM |
| L-met | — | 5 mM |
| % WW Ox | 96.6 | 12.3 |
| Binding to Ag3 | Impacted | 89 |
| (% relative potency) | | |

The addition of NAT+methionine to solution drastically reduced oxidation of Mab3.

### Example 3. Addition of Anti-Oxidants Mitigates Chemical Oxidation Risk

Mab4, an IgG1 antibody, was formulated at 100 mg/mL in 20 mM histidine HCl, 50 mM sodium chloride, 200 mM sucrose, 0.04% poloxamer 188. Antibody formulations were then incubated in the presence of AAPH at 0, 5, 10 mM or 10 mM AAPH+1 mM NAT+5 mM methionine at 40° C. for 24 hours. Samples were then evaluated by MS as described above.

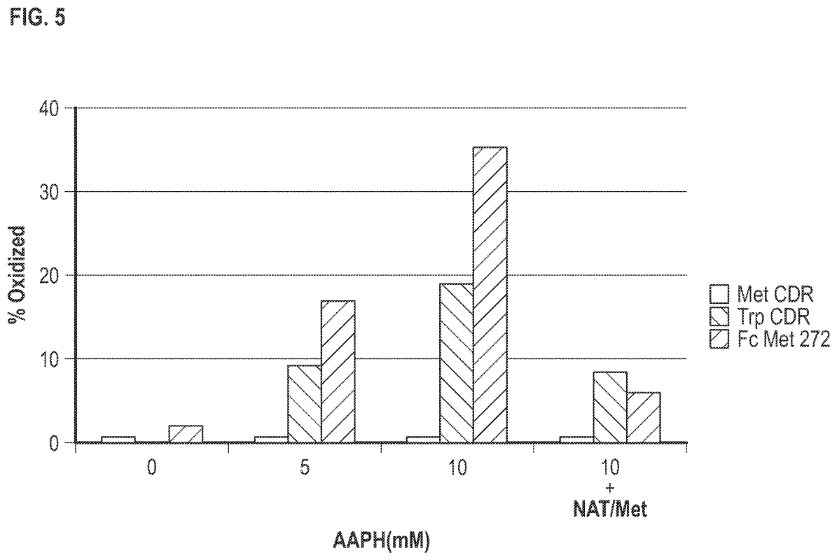

Results are show in FIG. 5. Approximately 15% oxidation of Fc M272 was observed at 5 mM AAPH. This corresponds to 10% trp oxidation. The addition of 1 mM NAT+5 mM methionine reduced oxidation by about 50% for Trp and about 80% for Fc Met 272. No change in Met CDR was observed at any level. Addition of NAT+met ameliorated the reduction in specific activity of Mab4 to bind Ag4 as measured by ELISA.

TABLE 2
Potency of antibodies

| AAPH | % specific activity |
|---|---|
| 0 | 106 |
| 5 | 63 |
| 10 | 43 |
| 10 + 1 mM NAT/5 mM methionine | 88 |

### Example 4. Addition of NAT/Met for Light Oxidiation Risk Mitigation

As study was conducted to determine if NAT/met can reduce light oxidation. Mab5, an IgG antibody having an isotype different from IgG1, was formulated at 150 mg/ml in 200 mM arginine succinate, pH 5.5 without NAT and met, with 0.3 mM NAT+5 mM methionine, or 0.3 mM NAT+10 mM methionine. Samples were exposed to 300,000 lux hours to assess risk. Results are presented in Table 3.

TABLE 3
Ambient light stress

| NAT/met level | treatment | Fc M251 | CDR-H3 W104 | HMWS | Color |
|---|---|---|---|---|---|
| 0 mM NAT/met | Foil (no light) | 3.0% | 1.6% | 0.80% | B5.3 |
| 0 mM NAT/met | Ambient | 15.5% | 6.1% | 1.30% | BY22 |
| 0.3 mM NAT/5 mM met | Ambient | 6.1% | 3.2% | 0.90% | B5.1 |
| 0.3 mM NAT/10 mM met | Ambient | 5.4% | 3.0% | 0.90% | B5.2 |

NAT/met protected Mab5 from ambient light related oxidation.

### Example 5. Addition of NAT/Met Provides Oxidation and Potency Protection

Antibodies drug products may show approximately 7-8% oxidation of Met251 at the end of shelf life, typically greater than 2 years at 5° C. To mimic this, antibodies were treated with 5 mM AAPH which yields about 15% oxidation of Met251. Samples were treated with 5 mM AAPH with or without NAT/met and then analyzed for oxidation of W104 and M251. Potency of antibodies was also measured. As shown in Table 4, addition of 0.3 mM NAT+5 mM methionine to a pool of antibodies reduced oxidation of W104 and M251 and reduced the decrease in potency of antibodies following AAPH stress.

TABLE 4
NAT/met provides oxidation and potency protection

| | NAT | Met | % W104 | % Fc M251 | |
|---|---|---|---|---|---|
| Material | (mM) | (mM) | oxidation | oxidation | Potency |
| Pool of | 0 | 0 | 36.6 | 12.2 | ~70 |
| clones | | | | | |
| Pool of | 0.3 | 5 | 26.4 | 4.6 | ~80 |
| clones | | | | | |

In addition, the pools of clones were subject to ambient light stress as described above. As shown in Table 5, the pools of clones experience the same color changes as described above.

TABLE 5
Light protection of antibodies

| Sample | Treatment | HMWS | Color |
|---|---|---|---|
| 0 mM NAT/met | Foil control | 0.71% | B5.3 |
| 0 mM NAT/met | Ambient light | 0.92% | BY3.0 |
| 0.3 mM NAT/5 mM met | Ambient light | 0.74% | B5.4 |

### Example 6. NAT/Met Mitigate Chemical Oxidation Risk

The AAPH stress test was used to assess oxidation protection by NAT and/or methionine. Mab6, a bispecific antibody, was incubated at 1 mg/mL in 20 mM histidine acetate with 1 mM AAPH for 16 hours at 40 C, with or without NAT and/or methionine. Samples were analyzed for oxidation as described above. As shown in Table X, NAT concentration of 0.1 to 0.5 m M provide oxidation protection, met alone also provides some protection from AAPH.

TABLE 6
NAT/met mitigate chemical oxidation risk

| | 1 | 2 | 3 | 4 | 5 | 6 |
|---|---|---|---|---|---|---|
| NAT | 0.1 mM | 0.4 mM | 0.5 mM | 0.3 mM | 0 mM | 0 mM |
| Met | 5 mM | 5 mM | 5 mM | 0 mM | 5 mM | 0 mM |
| W104 oxidation | 25.6% | 5.7% | 3.5% | 8.0% | 49.0% | 59.0% |

### Example 7. NAT/Met Protects Against Chemically-Induced Oxidation

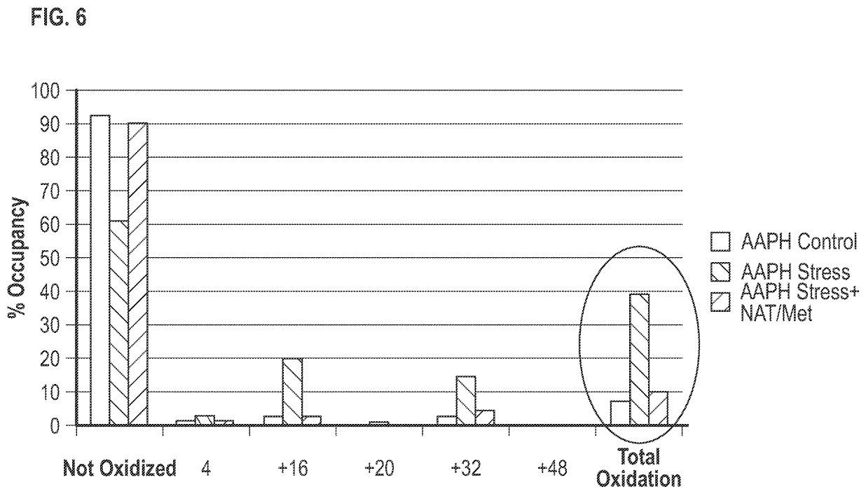

Antibody Mab7, a bispecific antibody, was evaluated for chemically-induced oxidation of position W52 by incubating the molecule at 1 mg/mL in 20 mM histidine acetate with 1 mM AAPH for 16 hours at 40° C., with or without NAT and methionine. Samples were analyzed by peptide map for oxidation. As shown in FIG. 6, the combination of NAT+met protected W52 from chemically induced oxidation.

What is claimed is:

1. A liquid formulation comprising a polypeptide, N-acetyl-DL-tryptophan (NAT), and L-methionine, wherein the NAT is provided in an amount sufficient to prevent oxidation of one or more tryptophan residues in the polypeptide, and wherein the L-methionine is provided in an amount sufficient to prevent oxidation of one or more methionine residues in the polypeptide.

2. The liquid formulation of claim 1, wherein the concentration of NAT in the formulation is about 0.01 to about 25 mM.

3. The liquid formulation of claim 1 or claim 2, wherein the concentration of NAT in the formulation is about 0.05 to about 1.0 mM.

4. The liquid formulation of any one of claims 1-3, wherein the concentration of NAT in the formulation is about 0.05 to about 0.3 mM.

5. The liquid formulation of any one of claims 1-4, wherein the concentration of NAT in the formulation is a concentration selected from the group consisting of about 0.05 mM, about 0.1 mM, about 0.3 mM, and about 1.0 mM.

6. The liquid formulation of any one of claims 1-5, wherein the concentration of L-methionine in the formulation is about 1 to about 125 mM.

7. The liquid formulation of any one of claims 1-6, wherein the concentration of L-methionine in the formulation is about 5 to about 25 mM.

8. The liquid formulation of any one of claims 1-7, wherein the concentration of L-methionine in the formulation is about 5 mM.

9. The liquid formulation of any one of claims 1-8, wherein the concentration of NAT in the formulation is about 0.3 mM and the concentration of L-methionine in the formulation is about 5.0 mM.

10. The liquid formulation of any one of claims 1-8, wherein the concentration of NAT in the formulation is about 1.0 mM and the concentration of L-methionine in the formulation is about 5.0 mM.

11. The liquid formulation of any one of claims 1-10, wherein the polypeptide is an antibody.

12. The liquid formulation claim 11, wherein the one or more tryptophan residues are located within a variable region of the antibody.

13. The liquid formulation of claim 11 or claim 12, wherein the one or more tryptophan residues comprises W103, wherein residue numbering is according to Kabat numbering.

14. The liquid formulation of any one of claims 11-13, wherein the one or more tryptophan residues are located within an HVR of the antibody.

15. The liquid formulation of any one of claims 11-14, wherein the one or more tryptophan residues are located within an HVR-H1 and/or an HVR-H3 of the antibody.

16. The liquid formulation of any one of claims 11-15, wherein the one or more tryptophan residues comprises W33, W36, W52a, W99, W100a, and/or W100b, wherein residue numbering is according to Kabat numbering.

17. The liquid formulation of any one of claims 11-16, wherein the one or more methionine residues are located within a variable region of the antibody.

18. The liquid formulation of any one of claims 11-17, wherein the one or more methionine residues comprises M34 and/or M82, wherein residue numbering is according to Kabat numbering.

19. The liquid formulation of any one of claims 11-18, wherein the one or more methionine residues are located within a constant region of the antibody.

20. The liquid formulation of any one of claims 11-19, wherein the one or more methionine residues comprises M252 and/or M428, wherein residue numbering is according to EU numbering.

21. The liquid formulation of any one of claims 11-20, wherein the antibody is an IgG1, IgG2, IgG3, or IgG4 antibody.

22. The liquid formulation of any one of claims 11-21, wherein the antibody is a polyclonal antibody, a monoclonal antibody, a humanized antibody, a human antibody, a chimeric antibody, a multispecific antibody, or an antibody fragment.

23. The liquid formulation of any one of claims 1-22, wherein the oxidation of the one or more tryptophan residues in the polypeptide is reduced relative to the oxidation of one or more corresponding tryptophan residues in the polypeptide in a liquid formulation lacking NAT.

24. The liquid formulation of any one of claims 1-23, wherein the oxidation of the one or more methionine residues in the polypeptide is reduced relative to the oxidation of one or more corresponding methionine residues in the polypeptide in a liquid formulation lacking L-methionine.

25. The liquid formulation of any one of claims 1-24, wherein the oxidation of the one or more tryptophan residues and the one or more methionine residues in the polypeptide is reduced relative to the oxidation of one or more corresponding tryptophan residues and one or more corresponding methionine residues in the polypeptide in a liquid formulation lacking NAT and L-methionine

26. The liquid formulation of any one of claims 23-25, where the oxidation is reduced by about 40%, about 50%, about 75%, about 80%, about 85%, about 90%, about 95% or about 99%.

27. The liquid formulation of any one of claims 1-26, wherein the polypeptide concentration in the formulation is about 1 mg/mL to about 250 mg/mL.

28. The liquid formulation of any one of claims 1-27, wherein the formulation has a pH of about 4.5 to about 7.0.

29. The liquid formulation of any one of claims 1-28, wherein the formulation further comprises one or more excipients.

30. The liquid formulation of claim 29, wherein the one or more excipients are selected from the group consisting of a stabilizer, a buffer, a surfactant, and a tonicity agent.

31. The liquid formulation of any one of claims 1-30, wherein the formulation is a pharmaceutical formulation suitable for administration to a subject.

32. The liquid formulation of claim 31, wherein the pharmaceutical formulation is suitable for subcutaneous, intravenous, or intravitreal administration.

33. The liquid formulation of claim 31 or claim 32, wherein the subject is a human.

34. An article of manufacture or kit comprising the liquid formulation of any one of claims 1-33.

35. A method of reducing oxidation of a polypeptide in an aqueous formulation comprising adding NAT and L-methionine to the formulation, wherein the NAT is provided in an amount sufficient to prevent oxidation of one or more tryptophan residues in the polypeptide, and wherein the L-methionine is provided in an amount sufficient to prevent oxidation of one or more methionine residues in the polypeptide.

36. The method of claim 35, wherein the NAT is added to the formulation to a concentration of about 0.01 to about 25 mM.

37. The method of claim 35 or claim 36, wherein the NAT is added to the formulation to a concentration of about 0.05 to about 1 mM.

38. The method of any one of claims 35-37, wherein the NAT is added to the formulation to a concentration of about 0.05 to about 0.3 mM.

39. The method of any one of claims 35-38, wherein the NAT is added to the formulation to a concentration selected from the group consisting of about 0.05 mM, about 0.1 mM, about 0.3 mM, and about 1.0 mM.

40. The method of any one of claims 35-39, wherein the L-methionine is added to the formulation to a concentration of about 1 to about 125 mM.

41. The method of any one of claims 35-40, wherein the L-methionine is added to the formulation to a concentration of about 5 to about 25 mM.

42. The method of any one of claims 35-41, wherein the L-methionine is added to the formulation to a concentration of about 5 mM.

43. The method of any one of claims 35-42, wherein the NAT is added to the formulation to a concentration of about 0.3 mM, and wherein the L-methionine is added to the formulation to a concentration of about 5.0 mM.

44. The method of any one of claims 35-42, wherein the NAT is added to the formulation to a concentration of about 1.0 mM, and wherein the L-methionine is added to the formulation to a concentration of about 5.0 mM.

45. The method of any one of claims 35-44, wherein the polypeptide is an antibody.

46. The method of claim 45, wherein the one or more tryptophan residues are located within a variable region of the antibody.

47. The method of claim 45 or claim 46, wherein the one or more tryptophan residues comprises W103, wherein residue numbering is according to Kabat numbering.

48. The method of any one of claims 45-47, wherein the one or more tryptophan residues are located within an HVR of the antibody.

49. The method of any one of claims 45-48, wherein the one or more tryptophan residues are located within an HVR-H1 and/or an HVR-H3 of the antibody.

50. The method of any one of claims 45-49, wherein the one or more tryptophan residues comprises W33, W36, W52a, W99, W100a, and/or W100b, wherein residue numbering is according to Kabat numbering.

51. The method of any one of claims 45-50, wherein the one or more methionine residues are located within a variable region of the antibody.

52. The method of any one of claims 45-51, wherein the one or more methionine residues comprises M34 and/or M82, wherein residue numbering is according to Kabat numbering.

53. The method of any one of claims 45-52, wherein the one or more methionine residues are located within a constant region of the antibody.

54. The method of any one of claims 45-53, wherein the one or more methionine residues comprises M252 and/or M428, wherein residue numbering is according to EU numbering.

55. The method of any one of claims 45-54, wherein the antibody is an IgG1, IgG2, IgG3, or IgG4 antibody.

56. The method of any one of claims 45-55, wherein the antibody is a polyclonal antibody, a monoclonal antibody, a humanized antibody, a human antibody, a chimeric antibody, a multispecific antibody, or an antibody fragment.

57. The method of any one of claims 35-56, wherein the oxidation of the one or more tryptophan residues in the polypeptide is reduced relative to the oxidation of one or more corresponding tryptophan residues in the polypeptide in a liquid formulation lacking NAT.

58. The method of any one of claims 35-57, wherein the oxidation of the one or more methionine residues in the polypeptide is reduced relative to the oxidation of one or more corresponding methionine residues in the polypeptide in a liquid formulation lacking L-methionine.

59. The method of any one of claims 35-58, wherein the oxidation of the one or more tryptophan residues and the one or more methionine residues in the polypeptide is reduced relative to the oxidation of one or more corresponding tryptophan residues and one or more corresponding methionine residues in the polypeptide in a liquid formulation lacking NAT and L-methionine.

60. The method of any one of claims 57-59, where the oxidation is reduced by about 40%, about 50%, about 75%, about 80%, about 85%, about 90%, about 95% or about 99%.

61. The method of any one of claims 35-60, wherein the polypeptide concentration in the formulation is about 1 mg/mL to about 250 mg/mL.

62. The method of any one of claims 35-61, wherein the formulation has a pH of about 4.5 to about 7.0.

63. The method of any one of claims 35-62, wherein the formulation further comprises one or more excipients.

64. The method of claim 63, wherein the one or more excipients are selected from the group consisting of a stabilizer, a buffer, a surfactant, and a tonicity agent.

65. The method of any one of claims 35-64, wherein the formulation is a pharmaceutical formulation suitable for administration to a subject.

66. The method of claim 65, wherein the pharmaceutical formulation is suitable for subcutaneous, intravenous, or intravitreal administration.

67. The method of claim 65 or claim 66, wherein the subject is a human.
